(19) United States
(12) Patent Application Publication
(10) Pub. No.: US 2017/0342493 A1
(43) Pub. Date: Nov. 30, 2017

(54) THERAPY SELECTION FOR PSORIASIS AND PSORIATIC ARTHRITIS

(71) Applicant: Fundació Hospital Universitari Vall D'Hebron-Institut De Recerca, Barcelona (ES)

(72) Inventors: Sara Marsal BARRIL, Barcelona (ES); Antonio Julià CANO, Barcelona (ES); Juan De Dios Canete CRESPILLO, Barcelona (ES); Carles Ferrándiz FORASTER, Badalona (ES)

(73) Assignees: Fundació Hospital Universitari Vall D'Hebron-Institut De Recerca, Barcelona (ES); Hospital Clinic De Barcelona, Barcelona (ES); Fundació Institut d'Investigació en Ciències de la Salut Germans Trias i Pujol, Badalona (ES)

(21) Appl. No.: 15/432,008

(22) Filed: Feb. 14, 2017

Related U.S. Application Data
(63) Continuation of application No. PCT/IB2015/056212, filed on Aug. 14, 2015.
(60) Provisional application No. 62/038,151, filed on Aug. 15, 2014.

(51) Int. Cl. C12Q 1/68; C07K 16/24; A61K 39/00

(52) U.S. Cl. CPC C12Q 1/6883; C07K 16/241; C12Q 2600/106; A61K 2039/505; C12Q 2600/156; C07K 2317/76

(57) ABSTRACT

The present invention provides methods for selecting an individual with psoriasis (Ps) or psoriatic arthritis (PsA) who should receive or who is likely to respond to a treatment with an anti-tumor necrosis factor alpha (anti-TNFα) therapy. In addition, provided herein are methods for selecting an individual with Ps or PsA who should receive or who is likely to respond to a therapy that is not an anti-TNFα therapy, e.g., a non-anti-TNFα therapy for the treatment of Ps or PsA. Specifically, the methods of the present invention relate to detecting the presence of distinct alleles of the PDE3A-SLCO1C1 locus which are associated with a clinical response to an anti-TNFα therapy or a non-anti-TNF therapy in patients with Ps or PsA.

### CROSS-REFERENCES TO RELATED APPLICATIONS

This application is a continuation of PCT Application No. PCT/IB2015/056212, filed Aug. 14, 2015, which claims priority to U.S. Provisional Application No. 62/038,151, filed Aug. 15, 2014, the disclosures of which are hereby incorporated by reference in their entirety for all purposes.

### BACKGROUND OF THE INVENTION

Psoriasis (e.g., psoriasis or psoriasis vulgaris) is a chronic inflammatory disease of the skin and is one of the most prevalent autoimmune diseases in the world (Nestle et al, N Engl J Med, 2009; 361(5): 496-509). It is a heterogeneous disease with a complex etiology, which includes the contribution of multiple risk genes. Genetic research studies in psoriasis, particularly through genome-wide association analysis, have identified more than 40 loci associated with the susceptibility to the disease (Nair et al., Nat Genet 2009; 41(2): 199-204; Ellinghaus et al., Nat Genet 2010; 42(11): 991-995; Huffmeier et al., Nat Genet 2010; 42(11): 996-999; Strange et al., Nat Genet 2010; 42(11): 985-990; Sun et al., Nat Genet 2010; 42(11): 1005-1009; Stuart et al., Nat Genet 2010; 42(11): 1000-1004; Tsoi et al., Nat Genet 2012; 44(12): 1341-1348). However, little is known about the genetic factors that influence key clinical aspects of the disease, such as a patient's response to therapy.

The chronic inflammatory process occurring in psoriasis skin lesions is characterized by the overexpression of multiple cytokines including tumor necrosis factor α (TNFα). TNFα is also a cytokine overexpressed in other chronic inflammatory diseases like rheumatoid arthritis, psoriatic arthritis or Crohn's disease (Feldmann, Nat Rev Immunol 2002; 2(5): 364-371). Similar to these other autoimmune diseases, the blockade of the levels of TNFα has proven to be a highly effective therapeutic approach to controlling the inflammatory process in psoriasis (Chaudhari et al., Lancet 2001; 357(9271): 1842-1847). However, a significant percentage of anti-TNFα-treated psoriasis patients (˜30%) do not show a significant clinical response (Prieto-Perez et al., Pharmacogenomics J 2013; 13(4): 297-305; Mahil et al., Br J Dermatol 2013; 169(2): 306-313). Anti-TNFα agents are costly and are not exempt of secondary effects (Smith et al., Br J Dermatol, 2009; 161(5): 987-1019).

Genetic variation not only contributes to the increase the risk to develop psoriasis but also it has been shown to influence the risk to developing different clinically relevant phenotypes (Julia et al., Hum Mol Genet 2012; 21(20): 4549-4557). Using this approach, the psoriasis risk locus TNFAIP3 (Nair et al., Nat Genet 2009; 41(2): 199-204; Strange et al., Nat Genet 2010; 42(11): 985-990) has been recently found to be associated with a clinical response to anti-TNFα therapy (Tejasvi et al., J Invest Dermatol 2012; 132(3 Pt 1): 593-600). In particular, it was found that the association was significant in the patients treated with etanercept, a TNF receptor fusion protein, but not with anti-TNFα monoclonal antibodies adalimumab and infliximab. This result suggests that the genetic variation associated to treatment response is not homogeneous for all treatments and adds a new layer of complexity to the analysis of psoriasis pharmacogenetics. Thus far, however, no other psoriasis risk gene has been associated to anti-TNFα drug response.

Psoriatic arthritis (PsA) is a chronic musculoskeletal inflammatory disease characterized by the presence of arthritis together with psoriasis. Originally thought to be a variant of rheumatoid arthritis (RA), PsA is now clearly considered a distinct disease entity (Gladman, J Rheumatol Suppl, 2012, 89, 106-110). The lack of rheumatoid factor, the presence of dactylitis, juxta-articular bone formation, nail dystrophy and a family history of psoriasis are distinctive features of PsA and are actually used as a diagnostic criteria for the disease (Taylor et al., Arthritis Rheum, 2006, 54(8), 2665-2673). Using these attributes, known as CASPAR criteria, PsA prevalence in psoriasis in large patient cohorts has been recently estimated to be about 12-14% (Ibrahim et al., Arthritis Rheum, 2009, 61(10), 1373-1378; Julia et al., Hum Mol Genet, 2012, 21(20), 4549-4557).

If inadequately controlled, the activity of PsA leads to the destruction of joints and to permanent disability (Kristensen et al., Ann Rheum Dis, 2013, 72(10), 1675-1679). In order to prevent disease progression, different treatment strategies are available (Gossec et al., Ann Rheum Dis, 2012, 71(1), 4-12). In particular, the systemic blockade of TNFα has proven to be the most effective in controlling moderate-to-severe PsA. Nonetheless, up to about 40% of the anti-TNFα treated patients do not show a significant clinical response to this therapy (Saad et al., Rheumatology, 2010, (Oxford) 49(4), 697-705; Kristensen et al., Ann Rheum Dis, 2008, 67(3), 364-369). Given the high cost of these drugs and the possibility of adverse events, there is a need to identify factors that can help predict the response to anti-TNFα agents. Clinical variables such as the degree of impairment of physical function, gender and the presence of glucocorticoid treatment have been studied in relation to treatment response (Van Den Bosch et al., Ann Rheum Dis, 2010 69(2), 394-399). However, the ability of these variables to predict treatment response in PsA is very limited and, therefore, new biomarkers are needed to improve treatment decisions (Villanova et al., Ann Rheum Dis, 2013, 72 Suppl 2, ii, 104-110).

PsA is a complex disease caused by the interaction of environmental and genetic risk factors (Chandran et al., Ann Rheum Dis, 2009, 68(5), 664-667). While the environmental contribution remains largely uncharacterized, there has recently been a significant advance in the characterization of the genetic basis of PsA. Genome-wide association studies have allowed the identification of multiple loci associated with psoriasis, most of which have also shown to be associated with the risk to develop PsA (Julia et al., Hum Mol Genet, 2012, 21(20), 4549-4557; Bowes et al., Ann Rheum Dis, 2011, 70, 1641-1644). GWAS performed in PsA patients exclusively have also found genetic factors independent from psoriasis (Ellinghaus et al., J Invest Dermatol, 2012, 132(4), 1133-1140). To date, however, the strongest risk factor for PsA resides in the HLA-C locus from the class I major histocompatibility complex in chromosome 6p21.3, which is also the main risk locus for psoriasis (Nair et al., Nat Genet, 2009, 41(2), 199-204). From a genetic susceptibility perspective, therefore, PsA and psoriasis share a substantial common genetic background.

Genetic variation not only has shown to increase the risk of developing PsA, but also to increase the risk of developing specific clinical features (Julia et al., Hum Mol Genet, 2012, 21(20), 4549-4557; O'Rielly and Rahman, Nat Rev Rheumatol, 2011, 7(12), 718-732). A patient's response to drugs, e.g., anti-TNFα agents, is a highly relevant clinical feature in PsA that strongly affects the course of the disease. To date, however, very few studies have analyzed the association of genetic variants with the response to anti-TNFα therapy in PsA (Prieto-Perez et al., Pharmacogenomics J 13(4), 297-305 (2013)).

As such, there is a need in the art for biomarkers, e.g., genetic biomarkers that aid or assist in determining the likelihood of clinical response to anti-TNFα treatment for psoriasis or psoriatic arthritis. The present invention satisfies this need and provides related advantages as well.

### BRIEF SUMMARY OF THE INVENTION

In one aspect, provided herein is a method for selecting an individual with psoriasis (Ps) to receive an anti-tumor necrosis factor α (anti-TNFα) therapy. The method comprises (a) detecting the presence of a single nucleotide polymorphism (SNP) in a PDE3A-SLCO1C1 locus in a sample obtained from the individual, wherein the SNP in the PDE3A-SLCO1C1 locus comprises rs3794271; and (b) selecting the individual to receive the anti-TNFα therapy based on the presence of a G allele or a complementary allele thereof at rs3794271. In some embodiments, step (b) of selecting the individual to receive the anti-TNFα therapy is based on the presence of two G alleles or the complementary alleles thereof at rs3794271.

In some embodiments, the anti-TNFα therapy is selected from the group consisting of infliximab (REMICADE™), etanercept (ENBREL™), adalimumab (HUMIRA™), certolizumab pegol (CIMZIA®), golimumab (SIMPONI®), ABT-122, pegsunercept, and combinations thereof. In other embodiments, the anti-TNFα therapy is selected from the group consisting of infliximab (REMICADE™), etanercept (ENBREL™) and adalimumab (HUMIRA™).

In some embodiments, the method further comprises detecting the presence of an allele of one or more SNPs in Table 1. In some embodiments, the method further comprises detecting the presence of a clinical factor for the individual. In some instances, the clinical factor is selected from the group consisting of age, gender, body mass index, nail dystrophy, erythrodermic phenotype, and combinations thereof. In some embodiments, the method further comprises applying a statistical analysis to the presence of the SNP in the PDE3A-SLCO1C1 locus, the presence of an allele of one or more SNPs in Table 1 and/or the presence of the clinical factor. The statistical analysis can improve the sensitivity, specificity, and/or overall accuracy of selecting the individual to receive the anti-TNFα therapy.

In some embodiments, the method further comprises selecting the individual to receive a therapy that is not an anti-TNFα therapy based on the presence of two A alleles or the complementary alleles thereof at rs3794271. In some embodiments, the therapy for Ps that is not an anti-TNFα therapy is selected from the group consisting of corticosteroids, vitamin D analogues, retinoids, anthralin, calcineurin inhibitors, salicylic acid, coal tar, phototherapy, methotrexate, cyclosporine, thioguanine, hydrourea, ustekinumab (STELARA®), A3 adenosine receptor agonists, anti-IL-17 agents, anti-IL-12/23 agents, anti-IL-17 receptor agents, Janus kinase (JAK) inhibitors, phosphodiesterase 4 (PDE4) inhibitors, biosimilars thereof, analogs thereof, derivative thereof, and combinations thereof.

In some embodiments, the sample is selected from the group consisting of whole blood, plasma, serum, synovial fluid, saliva, and urine. In some embodiments, the sample is whole blood, plasma or serum.

In another aspect, provided herein is a method for predicting a likelihood of response to an anti-tumor necrosis factor α (anti-TNFα) therapy in an individual with psoriasis (Ps). The method comprises (a) detecting the presence of a single nucleotide polymorphism (SNP) in a PDE3A-SLCO1C1 locus in a sample obtained from the individual, wherein the SNP in the PDE3A-SLCO1C1 locus comprises rs3794271; and (b) determining that the individual has a high likelihood of response to the anti-TNFα therapy based on the presence of a G allele or a complementary allele thereof at rs3794271. In some embodiments, step (b) of determining that the individual has the high likelihood of response to the anti-TNFα therapy is based on the presence of two G alleles or the complementary alleles thereof at rs3794271.

In some embodiments, the anti-TNFα therapy is selected from the group consisting of infliximab (REMICADE™), etanercept (ENBREL™), adalimumab (HUMIRA™), certolizumab pegol (CIMZIA®), golimumab (SIMPONI®), ABT-122, pegsunercept, and combinations thereof. In other embodiments, the anti-TNFα therapy is selected from the group consisting of infliximab (REMICADE™), etanercept (ENBREL™) and adalimumab (HUMIRA™).

In some embodiments, the method further comprises detecting the presence of an allele of one or more SNPs in Table 1. In some instances, the method further comprises detecting the presence of a clinical factor for the individual. The clinical factor may be selected from the group consisting of age, gender, body mass index, nail dystrophy, erythrodermic phenotype, and combinations thereof. In some embodiments, the statistical analysis improves the sensitivity, specificity, and/or overall accuracy of predicting the likelihood of response to the anti-TNFα therapy.

In some embodiments, the method further comprises determining that the individual has the high likelihood of response to a therapy that is not an anti-TNFα therapy based on the presence of two A alleles or the complementary alleles thereof at rs3794271. In some embodiments, the therapy for Ps that is not an anti-TNFα therapy is selected from the group consisting of corticosteroids, vitamin D analogues, retinoids, anthralin, calcineurin inhibitors, salicylic acid, coal tar, phototherapy, methotrexate, cyclosporine, thioguanine, hydrourea, ustekinumab (STELARA®), A3 adenosine receptor agonists, anti-IL-17 agents, anti-IL-12/23 agents, anti-IL-17 receptor agents, Janus kinase (JAK) inhibitors, phosphodiesterase 4 (PDE4) inhibitors, biosimilars thereof, analogs thereof, derivative thereof, and combinations thereof.

In some embodiments, the sample is selected from the group consisting of whole blood, plasma, serum, synovial fluid, saliva, and urine. In other embodiments, the sample is whole blood, plasma or serum.

In yet another aspect, provided herein is a method for selecting an individual with psoriatic arthritis (PsA) to receive an anti-TNFα therapy. The method includes (a) detecting the presence of a single nucleotide polymorphism (SNP) in a PDE3A-SLCO1C1 locus in a sample obtained from the individual, wherein the SNP in the PDE3A-SLCO1C1 locus comprises rs3794271; and (b) selecting the individual to receive the anti-TNFα therapy based on the presence of two A alleles or the complementary alleles thereof at rs3794271.

In some embodiments, the anti-TNFα therapy is selected from the group consisting of infliximab (REMICADE™), etanercept (ENBREL™), adalimumab (HUMIRA™), certolizumab pegol (CIMZIA®), golimumab (SIMPONI®), ABT-122, pegsunercept, and combinations thereof. In other embodiments, the anti-TNFα therapy is selected from the group consisting of infliximab (REMICADE™), etanercept (ENBREL™) and adalimumab (HUMIRA™).

In some embodiments, the method further comprises detecting the presence of an allele of one or more SNPs in Table 1. In other embodiments, the method further comprises detecting the presence of a clinical factor for the individual. In some instances, the clinical factor is selected from the group consisting of age, gender, HLA-B27 genotype, disease duration, dactylitis, enthesitis, axial involvement of PsA, nail dystrophy, severity of skin disease, and combinations thereof. In some embodiments, the method further comprises applying a statistical analysis to the presence of the SNP in the PDE3A-SLCO1C1 locus, the presence of an allele of one or more SNPs in Table 1 and/or the presence of the clinical factor. The statistical analysis can improve the sensitivity, specificity, and/or overall accuracy of selecting the individual to receive the anti-TNF therapy.

In some embodiments, the sample is selected from the group consisting of whole blood, plasma, serum, synovial fluid, saliva, and urine. In other embodiments, the sample is whole blood, plasma or serum.

In some embodiments, the method disclosed herein further includes selecting the individual with PsA to receive therapy for PsA that is not an anti-TNFα therapy based on the presence of at least a G allele (i.e., one G allele) or a complementary allele thereof at rs3794271.

In some cases, the step of selecting the individual to receive the therapy for PsA that is not an anti-TNFα therapy is based on the presence of two G alleles or the complementary alleles thereof at rs3794271.

In some embodiments, the therapy for PsA that is not an anti-TNFα therapy is selected from the group consisting of ustekinumab (STELARA®), bimekizumab, ixekizumab, sirukumab, sarilumab, secukinumab (COSENTYX®), ocrelizumab, rituximab)(Rituxin®), tocilizumab (ACTEMRA®, ofatumumab (ARZERRA®), denosumab (XGEVA®), abatacept (ORENCIA®, masitinib, baricitinib, tofacitinib (XEJLANZ®), anakinra (KINERET®), ABP 501 (Amgen), liraglutide, DMARDs, NSAIDs, PDE4 inhibitors, retinoids, glucocorticoids, immunosuppressive drugs, biosimilars thereof, derivatives thereof, analogs thereof, and combinations thereof. In other embodiments, the therapy for PsA that is not an anti-TNFα therapy is selected from the group consisting of DMARDs, NSAIDs, PDE4 inhibitors, retinoids, glucocorticoids, immunosuppressive drugs, biosimilars thereof, derivatives thereof, analogs thereof, and combinations thereof.

In some aspects, provided herein is a method for predicting a likelihood of response to an anti-tumor necrosis factor α (anti-TNFα) therapy in an individual with psoriatic arthritis (PsA). The method comprises (a) detecting the presence of a single nucleotide polymorphism (SNP) in a PDE3A-SLCO1C1 locus in a sample obtained from the individual, wherein the SNP in the PDE3A-SLCO1C1 locus comprises rs3794271; and (b) determining that the individual has a high likelihood of response to the anti-TNFα therapy based on the presence of two A alleles or complementary alleles thereof at rs3794271.

In some embodiments, the anti-TNFα therapy is selected from the group consisting of infliximab (REMICADE™), etanercept (ENBREL™), adalimumab (HUMIRA™), certolizumab pegol (CIMZIA®), golimumab (SIMPONI®), ABT-122, pegsunercept, and combinations thereof. In other embodiments, the anti-TNFα therapy is selected from the group consisting of infliximab (REMICADE™), etanercept (ENBREL™), and adalimumab (HUMIRA™).

In some embodiments, the method further comprises detecting the presence of an allele of one or more SNPs in Table 1. In other embodiments, the method also comprises detecting the presence of a clinical factor for the individual. The clinical factor may be selected from the group consisting of age, gender, HLA-B27 genotype, disease duration, dactylitis, enthesitis, axial involvement of PsA, nail dystrophy, severity of skin disease, and combinations thereof. In some embodiments, the method further comprises applying a statistical analysis to the presence of the SNP in the PDE3A-SLCO1C1 locus, the presence of an allele of one or more SNPs in Table 1 and/or the presence of the clinical factor. The statistical analysis may improve the sensitivity, specificity, and/or overall accuracy of predicting the likelihood of a response to the anti-TNFα therapy.

In some embodiments, the sample is selected from the group consisting of whole blood, plasma, serum, synovial fluid, saliva, and urine. In other embodiments, the sample is whole blood, plasma or serum.

In some embodiments, the method further comprises determining that the individual with PsA has a high likelihood of response to a therapy for PsA that is not an anti-TNFα therapy based on the presence of a G allele or a complementary allele thereof at rs3794271. In some cases, the individual has two G alleles or complementary alleles thereof at rs3794271.

In some embodiments, the therapy for PsA that is not an anti-TNFα therapy is selected from the group consisting of ustekinumab (STELARA®), bimekizumab, ixekizumab, sirukumab, sarilumab, secukinumab (COSENTYX®), ocrelizumab, rituximab (Rituxin®), tocilizumab (ACTEMRA®), ofatumumab (ARZERRA®), denosumab (XGEVA®), abatacept (ORENCIA®), masitinib, baricitinib, tofacitinib (XEJLANZ®), anakinra (KINERET®), ABP 501 (Amgen), liraglutide, DMARDs, NSAIDs, PDE4 inhibitors, retinoids, glucocorticoids, immunosuppressive drugs, biosimilars thereof, derivatives thereof, analogs thereof, and combinations thereof. In some embodiments, the therapy for PsA that is not an anti-TNFα therapy may be selected from the group consisting of DMARDs, NSAIDs, PDE4 inhibitors, retinoids, glucocorticoids, immunosuppressive drugs, biosimilars thereof, derivatives thereof, analogs thereof, and combinations thereof.

In other aspects, provided herein is a method for treating a human subject having psoriasis (Ps) comprising administering a therapeutically effective amount of an anti-TNFα therapy to a human subject suffering from Ps and having a G allele or a complementary allele thereof at rs3794271 in a PDE3A-SLCO1C1 locus. In some instances, the human subject has two G alleles or complementary alleles thereof at rs3794271.

In some cases, the anti-TNFα therapy is selected from the group consisting of infliximab (REMICADE™), etanercept (ENBREL™), adalimumab (HUMIRA™), certolizumab pegol (CIMZIA®), golimumab (SIMPONI®), ABT-122, pegsunercept, and combinations thereof. In some embodiments, the anti-TNFα therapy is infliximab (REMICADE™), etanercept (ENBREL™), or adalimumab (HUMIRA™).

In some aspects, provided herein is a method for treating a human subject having psoriasis (Ps) comprising administering a therapeutically effective amount of a therapy for Ps that is not an anti-TNFα therapy to a human subject suffering from Ps and having two A alleles or complementary alleles thereof at rs3794271 in a PDE3A-SLCO1C1 locus.

In some embodiments, the therapy for Ps that is not an anti-TNFα therapy is selected from the group consisting of corticosteroids, vitamin D analogues, retinoids, anthralin, calcineurin inhibitors, salicylic acid, coal tar, phototherapy, methotrexate, cyclosporine, thioguanine, hydrourea, ustekinumab (STELARA®), A3 adenosine receptor agonists, anti-IL-17 agents, anti-IL-12/23 agents, anti-IL-17 receptor agents, Janus kinase (JAK) inhibitors, phosphodiesterase 4 (PDE4) inhibitors, biosimilars thereof, analogs thereof, derivative thereof, and combinations thereof.

In yet other aspects, provided herein is a method for treating a human subject having psoriatic arthritis (PsA) comprising administering a therapeutically effective amount of an anti-TNFαtherapy to a human subject suffering from PsA and having two A alleles or complementary alleles thereof at rs3794271 in a PDE3A-SLCO1C1 locus.

In some embodiments, the anti-TNFα therapy is selected from the group consisting of infliximab (REMICADE™), etanercept (ENBREL™), adalimumab (HUMIRA™), certolizumab pegol (CIMZIA®), golimumab (SIMPONI®), ABT-122, pegsunercept, and combinations thereof. In other embodiments, the anti-TNFα therapy is infliximab (REMICADE™), etanercept (ENBREL™), or adalimumab (HUMIRA™).

In some aspects, provided herein is a method for treating a human subject having psoriatic arthritis (PsA) comprising administering a therapeutically effective amount of a therapy for PsA that is not an anti-TNFα therapy to a human subject suffering from PsA and having a G allele or a complementary allele thereof at rs3794271 in a PDE3A-SLCO1C1 locus. In some embodiments, the human subject has two G alleles or the complementary alleles thereof at rs3794271.

In some embodiments, the therapy for PsA that is not an anti-TNFα therapy is selected from the group consisting of ustekinumab (STELARA®), bimekizumab, ixekizumab, sirukumab, sarilumab, secukinumab (COSENTYX®), ocrelizumab, rituximab (Rituxin®), tocilizumab (ACTEMRA®), ofatumumab (ARZERRA®), denosumab (XGEVA®), abatacept (ORENCIA®), masitinib, baricitinib, tofacitinib (XEJLANZ®), anakinra (KINERET®), ABP 501 (Amgen), liraglutide, DMARDs, NSAIDs, PDE4 inhibitors, retinoids, glucocorticoids, immunosuppressive drugs, biosimilars thereof, derivatives thereof, analogs thereof, and combinations thereof. In other embodiments, the therapy for PsA that is not an anti-TNFα therapy is selected from the group consisting of DMARDs, NSAIDs, PDE4 inhibitors, retinoids, glucocorticoids, immunosuppressive drugs, biosimilars thereof, derivatives thereof, analogs thereof, and combinations thereof.

Other objects, features, and advantages of the present invention will be apparent to one of skill in the art from the following detailed description and FIGURE.

### BRIEF DESCRIPTION OF THE DRAWINGS

FIG. 1 shows the interaction of PDE3A-SLCO1C1 genotype with the response to anti-TNFα therapy according to gender. The change in DAS28 score (ΔDAS28) between week 0 and 12 of anti-TNFα therapy (y-axis) is plotted according to the presence of a G allele at rs3794271 SNP (x-axis). Male (black) and female (grey) ΔDAS28 values are connected by a line. The change in slope between both gender subgroups clearly shows the presence of a strong interaction with the variation at PDE3A-SLCO1C1.

### DETAILED DESCRIPTION OF THE INVENTION

### I. Introduction

The present invention is based, in part, on the discovery of a specific allelic variant at the PDE3A-SLCO1C1 locus, e.g., rs3794271 that is useful for predicting whether a subject with psoriasis (Ps) or psoriatic arthritis (PsA) will respond to an anti-TNFα drug. In some aspects, the presence of one or more G alleles or the complementary allele(s) thereof at rs3794271 can be used to select a subject with Ps who should receive anti-TNFα therapy. On the other hand, a subject with Ps and having an AA genotype at rs3794271 will likely have a reduced or poor response to an anti-TNFα therapy, and a positive response to a therapy for Ps that is not an anti-TNFα drug. In other aspects, the presence of two A alleles or the complementary alleles thereof at rs3794271 can be used to identify a subject with PsA who is likely to have a positive response to anti-TNFα therapy. As such, a subject with PsA and having two A alleles or the complementary alleles thereof at rs3794271 can be administered an anti-TNFα therapy. On the other hand, a subject with PsA and having a G or GG genotype at rs3794271 will likely have a reduced or poor response to an anti-TNFα therapy, and a positive response to a therapy for PsA that is not an anti-TNFα drug.

rs3794271 corresponds to an A/G SNP (or the complement thereof such as a T/C SNP) located in the fourth intron of the SLCO1C1 gene (Gene ID No. 53919) at chromosome 12p12.2. The rs3794271 polymorphic site is located at chromosomal position of 20707159 of GRch38, which is position 13622509 of contig NT_009714.18.

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “psoriasis” or “Ps” refers to a skin disease characterized by hyperplasia of keratinocytes resulting in thickening of the epidermis and the presence of red scaly plaques. Guttate psoriasis, with raindrop shaped lesions scattered on the trunk and limbs, is the most frequent form in children, while pustular psoriasis is usually localized to the palms and soles. Erythrodermic psoriasis is an inflamed lesion of the skin with fine scales, and is frequently accompanied by severe pain, itching, and possibly swelling. Plaque psoriasis, the most commonly observed variety, is characterized by inflamed lesions covered with a silvery white scale. Although plaque psoriasis may occur on any skin surface, it is most commonly found on the knees, elbows, scalp, and trunk. The classical inflammatory lesions vary from discrete erythematous papules and plaques covered with silvery scales, to scaly itching patches that bleed when the scales are removed. Psoriasis may also present itself as pits in toenails and fingernails. The pitting may be accompanied by discoloration and thickening of the nail, and the nail may detach from the nail bed.

The term “psoriatic arthritis” or “PsA” includes chronic inflammatory arthritic condition that affects the skin, the joints, the insertion sites of tendons, ligaments, and fascia. Psoriatic arthritis is commonly associated with psoriasis. Approximately 6-42% of patients with psoriasis develop psoriatic arthritis. The disease typically appears between the ages of 30-55 but it can be diagnosed during childhood. Men and women have an equal risk for developing the condition. Symptoms of psoriatic arthritis include extra bone formation, joint stiffness, dactylitis, enthesopathy, tendonitis, and spondylitis. Most patients have the classic psoriasis pattern of skin lesions. Scaly, erythematous plaques; guttate lesions, lakes of pus, and erythroderma are psoriatic skin lesions that may be seen in patients with psoriatic arthritis. Nail lesions, including pitting, Beau lines, leukonychia, onycholysis, oil spots, subungual hyperkeratosis, splinter hemorrhages, spotted lunulae, and cracking, are clinical features significantly associated with the development of psoriatic arthritis. Ocular symptoms in psoriatic arthritis include conjunctivitis, iritis, episcleritis, keratoconjunctivitis sicca and aortic insufficiency.

There are about 5 types of psoriatic arthritis: symmetric, asymmetric, distal interphlangeal predominant, spondylitis, and arthritis mutilans. Symmetric PsA accounts for about 50% of the cases and affects joints on both sides of the body at the same time. Asymmetric PsA affects about 35% of the patients with PsA. Inflammation and stiffness at the ends of the fingers and toes are characteristic symptoms of distali interphalangeal predominant. Patients with spondylitis PsA exhibit pain and stiffness in the spine and neck. Arthritis mutilans is a considered the most severe form of PsA and causes deformities in the small joints at the ends of the fingers and toes.

The term “PDE3A-SLCO1C1 locus” refers to the genomic region on human chromosome 12 that includes the PDE3A gene, the 5 the SLCO1C1 gene, and the flanking genomic regions at the 5′ end and 3′ end of either genes.

The term “subject,” “patient,” or “individual” typically includes humans, but can also include other animals such as, e.g., other primates, rodents, canines, felines, equines, ovines, porcines, and the like. In some embodiments, the term “subject” refers to a human subject suffering from psoriasis and in need of treatment. In other embodiments, the term “subject” refers to a human subject suffering from psoriatic arthritis and in need of treatment.

The term “sample” includes any biological specimen obtained from an individual. Suitable samples for use in the present invention include, without limitation, whole blood, plasma, serum, synovial fluid, saliva, urine, stool, tears, any other bodily fluid, tissue samples (e.g., biopsy), and cellular extracts thereof (e.g., red blood cellular extract). In a preferred embodiment, the sample is a whole blood, serum or plasma sample. The use of samples such as serum, saliva, and urine is well known in the art (see, e.g., Hashida et al., J. Clin. Lab. Anal., 11:267-86 (1997)). One skilled in the art will appreciate that samples such as plasma or serum samples can be diluted prior to the analysis of marker levels. The term “bodily fluid” refers to a liquid originating from inside the living body. It includes fluids that are excreted or secreted from the body as well as those that normally are not.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region, such as the promoter and 3′-untranslated region, respectively, as well as intervening sequences (introns) between individual coding segments (exons).

The term “genotype” refers to the genetic composition of an organism, including, for example, whether a diploid organism is heterozygous or homozygous for one or more variant alleles of interest.

The term “polymorphism” refers to the occurrence of two or more genetically determined alternative sequences or alleles in a population. A “polymorphic site” refers to the locus at which divergence occurs. Preferred polymorphic sites have at least two alleles, each occurring at a particular frequency in a population. A polymorphic locus may be as small as one base pair (i.e., single nucleotide polymorphism or SNP). Polymorphic markers include restriction fragment length polymorphisms, variable number of tandem repeats (VNTR's), hypervariable regions, mini satellites, dinucleotide repeats, trinucleotide repeats, tetranucleotide repeats, simple sequence repeats, and insertion elements such as Alu. The first identified allele is arbitrarily designated as the reference allele, and other alleles are designated as alternative alleles, “variant alleles,” or “variances.” The allele occurring most frequently in a selected population is sometimes referred to as the “wild-type” allele or “major” allele. Diploid organisms may be homozygous or heterozygous for the variant alleles. The variant allele may or may not produce an observable physical or biochemical characteristic (“phenotype”) in an individual carrying the variant allele. For example, a variant allele may alter the enzymatic activity of a protein encoded by a gene of interest.

The term “single nucleotide polymorphism” or “SNP” refers to a change of a single nucleotide with a polynucleotide, including within an allele. This can include the replacement of one nucleotide by another, as well as deletion or insertion of a single nucleotide. Single nucleotide polymorphisms may fall within coding sequences of genes, non-coding regions of genes, or in the intergenic regions between genes. SNPs that are not in protein-coding regions may still have consequences for gene splicing, transcription factor binding, or the sequence of non-coding RNA. Most typically, SNPs are biallelic markers, although tri- and tetra-allelic markers can also exist. By way of non-limiting example, a nucleic acid molecule comprising SNP A\C may include a C or A or the complementary alleles thereof at the polymorphic position. For combinations of SNPs, the term “haplotype” is used, e.g. the genotype of the SNPs in a single DNA strand that are linked to one another. In some embodiments, the term “haplotype” can be used to describe a combination of SNP alleles, e.g., the alleles of the SNPs found together on a single DNA molecule. In further embodiments, the SNPs in a haplotype can be in linkage disequilibrium with one another. As there are for genes, there are also bioinformatics databases for SNPs. dbSNP is a SNP database from National Center for Biotechnology Information (NCBI).

The term “risk allele” refers to an allelic variant for a gene that is associated with an increased risk or likelihood of having a specific disease, disorder or condition.

The term “major allele” refers to an allele with the highest frequency at a locus that is observed in a population. For instance, the major allele can be the greater of the two allele frequencies for a single nucleotide polymorphism (SNP).

The term “minor allele” refers to an allele with the lowest frequency at a locus that is observed in a population. For instance, the minor allele can be the lesser of the two allele frequencies for a single nucleotide polymorphism (SNP). There are variations between human populations, so a SNP allele that is common in one geographical or ethnic group may be much rarer, or even absent, in another.

The term “linkage disequilibrium” or “LD” refers to any degree of non-random genetic association between one or more allele(s) of two different polymorphic DNA sequences and that is due to the physical proximity of the two loci. The term can refer to the trend for alleles at nearby loci on haploid genomes to correlate in the population. For example, a and b, alleles at close loci A and B, are said to be in linkage disequilibrium if the a b haplotype (a haplotype is defined as a set of alleles on the same chromosomal segment) has a frequency which is statistically higher than Pₐ×P_{b} (expected frequency if the alleles segregate independently, where Pₐ is the frequency of allele a, and P_{b} that of allele b). LD can be measured using the r² statistic.

The term “clinical factor” refers to a physiological attribute that at a certain level may be associated with an increased risk of a specific disease, disorder or condition. Non-limiting examples of clinical factors include patient age, gender, smoking history, family history of the disease, disease duration, disease activity, presence and severity of disease symptoms, genotype, etc.

The term “TNFα,” “tumor necrosis factor-α,” or “tumor necrosis factor,” as used herein, is intended to refer to a human cytokine that exists as a 17 kD secreted form and a 26 kD membrane associated form, the biologically active form of which is composed of a trimer of noncovalently bound 17 kD molecules. The structure of TNFα is described further in, for example, Pennica et al. (Nature 1984 312:724-729), Davis et al. (Biochemistry 1987 26:1322-1326) and Jones et al. (Nature 1989 338:225-228). The term TNFα is intended to include recombinant TNFα molecules, which can be prepared by standard recombinant expression methods or purchased commercially, as well as fusion proteins containing a TNFα molecule. Amino acid sequences of exemplary TNFαs that may be employed herein are found in the NCBI's Genbank database and a full description of human TNFα and its role in various diseases and conditions is found in NCBI's Online Mendelian Inheritance in Man database.

The term “anti-TNFα therapy” or “anti-TNFα drug” refers to an agent including peptides, proteins, antibodies, antibody fragments, fusion proteins, multivalent binding proteins, small molecule TNFα antagonists, similar naturally- or nonnaturally-occurring molecules, and/or recombinant and/or engineered forms thereof that inhibit/neutralize TNFα activity. The term “anti-TNF therapy” or “anti-TNF drug” includes an agent that inhibits the interaction of TNFα with a cell surface receptor for TNFα, inhibits TNFα protein production, inhibits TNFα gene expression, inhibits TNFα secretion from cells, inhibits TNFα receptor signaling or any other means resulting in decreased TNFα activity in a subject. Non-limiting examples of anti-TNFα drugs include infliximab (REMICADE™, Johnson and Johnson), adalimumab (D2E7/HUMIRA™, Abbott Laboratories), etanercept (ENBREL™, Amgen), pegsunercept (Amgen), ABT-122 (AbbVie), certolizumab pegol (CIMZIA®, UCB, Inc.), golimumab (SIMPONI®; CNTO 148), CDP 571 (Celltech), CDP 870 (Celltech), TRX 4 (TolerRx), as well as other compounds which inhibit/neutralize TNFα activity. The inhibition of the biological activity of TNFα can be assessed by measuring one or more indicators of TNFα biological activity, such as TNFα-induced cytotoxicity (either in vitro or in vivo), TNFα-induced cellular activation or TNFα binding to a TNFα receptor. TNFα biological activity can be assessed by one or more of several standard in vitro or in vivo assays known in the art.

The term “therapy for treating psoriasis (Ps) that is not an anti-TNF therapy” refers to an agent (e.g., oligonucleotide, inhibitory RNA, peptide, protein, antibody, antibody fragment, fusion proteins, multivalent binding proteins, small molecule, chemical compound, and the like that does not inhibit/neutralize TNFα activity and is useful for treating psoriasis. Non-limiting examples of a therapy for treating Ps that is not an anti-TNF therapy include corticosteroids, vitamin D analogues, retinoids, anthralin, calcineurin inhibitors, salicylic acid, coal tar, phototherapy, e.g., ultraviolet light therapy, UVB phototherapy, narrow band UVB therapy, Goeckerman therapy, photochemotherapy, and excimer laser therapy, methotrexate, cyclosporine, thioguanine, hydrourea, ustekinumab (STELARA®), A3 adenosine receptor agonists, anti-IL-17 agents, anti-IL-12/23 agents, anti-IL-17 receptor agents, Janus kinase (JAK) inhibitors, phosphodiesterase 4 (PDE4) inhibitors, biosimilars thereof, analogs thereof, derivative thereof, and combinations thereof.

The term “therapy for treating psoriatic arthritis (PsA) that is not an anti-TNF therapy” refers to an agent (e.g., oligonucleotide, inhibitory RNA, peptide, protein, antibody, antibody fragment, fusion proteins, multivalent binding proteins, small molecule, chemical compound, and the like that does not inhibit/neutralize TNFα activity and is useful for treating psoriatic arthritis. Non-limiting examples of a therapy for treating PsA that is not an anti-TNF therapy include ustekinumab (STELARA®), bimekizumab, ixekizumab, sirukumab, sarilumab, secukinumab (COSENTYX®), ocrelizumab, rituximab (Rituxin®), tocilizumab (ACTEMRA®), ofatumumab (ARZERRA®), denosumab (XGEVA®), abatacept (ORENCIA®), masitinib, baricitinib, tofacitinib (XEJLANZ®), anakinra (KINERET®), ABP 501 (Amgen), liraglutide, a disease-modifying anti-rheumatic drug (DMARD), a nonsteroidal anti-inflammatory drug (NSAID), a phosphodiesterase 4 (PDE4) inhibitor, a retinoid, a glucocorticoids, an immunosuppressive drug, free bases thereof, pharmaceutically acceptable salts thereof, derivatives thereof, analogs thereof, and any combinations thereof. Non-limiting examples of DMARDs include methotrexate, leflunomide, D-penicillamine, azathioprine, gold salts (e.g., sodium aurothiomalate, auranofin, etc.), cyclosporine, minocycline, anti-malarial medications (e.g., chloroquine, hydroxychloroquine, sulfasalazine, etc.), free bases thereof, pharmaceutically acceptable salts thereof, derivatives thereof, analogs thereof, and combinations thereof. Examples of NSAIDs include, but are not limited to, ibuprofen, indomethacin, COX-2 inhibitors (e.g., celecoxib), free bases thereof, pharmaceutically acceptable salts thereof, derivatives thereof, analogs thereof, and combinations thereof. Non-limiting examples of PDE4 inhibitors include apremilast, cilomilast, diazepam, ibudilast, luteolin, mesembrenone, piclamilast, roflumilast, rolipram, and combinations thereof.

The term “higher likelihood” or “high likelihood” in the context of a response to treatment refers to an above-average likelihood (chance or probability) that an individual will have a positive response to a treatment.

The term “lower likelihood” or “low likelihood” in the context of a response to treatment refers to an below-average likelihood (chance or probability) that an individual will have a positive response to a treatment.

The term “positive response” with respect to a therapeutic treatment refers to at least a partial marked reduction in the severity, an amelioration of one or more symptoms of a patient's disease or disorder, or a decrease or delay of disease progression.

The term “treat,” “treating” or “treatment” refers to an action that reduces the severity or symptoms of the disease or disorder, or retards or slows the progression or symptoms of the disease or disorder in a patient is suffering from the specified disease or disorder.

The term “therapeutically effective amount or dose” includes a dose of a drug (e.g., a PDE4 inhibitor) that is capable of achieving a therapeutic effect in a subject in need thereof. For example, a therapeutically effective amount of a drug useful for treating RA or PsA can be the amount that is capable of preventing or relieving one or more symptoms associated with RA or PsA. The exact amount can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “biosimilar” refers to a pharmaceutical agent or drug that is approved for clinical use based on evidence that it is similar, e.g., structurally and functionally similar, to a reference, approved pharmaceutical agent or drug and has not clinically meaningful differences in safety and effectiveness compared to the reference agent or drug.

### III. Detailed Description of the Embodiments

A. Therapy Selection for Psoriasis

The present invention provides a method for selecting or recommending a therapy regimen for an individual with psoriasis (Ps). In particular, the method can be used to identify an individual with Ps who should be administered an anti-TNFα therapy. Also provided is a method for determining the likelihood of response to an anti-TNFα therapy in an individual with Ps. In some embodiments, the method includes detecting the presence of a specific allelic variant at a SNP in the PDE3A-SLCO1C1 locus corresponding to rs3794271 in a biological sample obtained from an individual having Ps. If the presence of a G allele or a complementary allele thereof (i.e., C allele) is determined, the individual should be administered an anti-TNF therapy and/or is likely to respond to an anti-TNFα therapy. For instance, patients with one or two copies of a G allele (i.e., an AG or GG genotype) at rs3794271 are associated with a high likelihood of a positive response to an anti-TNFα drug compared to patients with two copies of an A allele of the same polymorphic site. A patient suffering from Ps and having at least one G allele or a complementary allele thereof at rs3794271 has an increased likelihood of responding to an anti-TNFα drug compared to a Ps patient with two A alleles or complementary alleles thereof at rs3794271.

In some embodiments, an individual having psoriasis and an AA genotype at a SNP in the PDE3A-SLCO1C1 locus such as rs3794271 is predicted to have a high likelihood of response to a therapy for treating Ps that is not an anti-TNFα therapy. As such, a therapy that is not an anti-TNFα therapy can be selected or recommended for a Ps subject having two AA alleles at rs3794271. In some embodiments, an individual diagnosed with Ps and homozygous for an A allele at rs3794271 is administered a therapy that is not an anti-TNFα therapy for the treatment of Ps. In some cases, a therapy for treating Ps that is not an anti-TNF therapy includes corticosteroids, vitamin D analogues, retinoids, anthralin, calcineurin inhibitors, salicylic acid, coal tar, phototherapy, e.g., ultraviolet light therapy, UVB phototherapy, narrow band UVB therapy, Goeckerman therapy, photochemotherapy, and excimer laser therapy, methotrexate, cyclosporine, thioguanine, hydrourea, ustekinumab (STELARA®), A3 adenosine receptor agonists, anti-IL-17 agents, anti-IL-12/23 agents, anti-IL-17 receptor agents, Janus kinase (JAK) inhibitors, phosphodiesterase 4 (PDE4) inhibitors, biosimilars thereof, analogs thereof, derivative thereof, and combinations thereof.

In some embodiments, the presence of at least one neighboring SNPs of rs3794271 that are in moderate to high linkage disequilibrium (e.g., r²=0.5, the lower threshold for moderate LD; r² refers to the square correlation coefficient) to the PDE3A-SLCO1C1 SNP is also detected. In some instances, the presence of one or more neighboring SNPs, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or more neighboring SNPs, of rs3794271 that are in high linkage disequilibrium to rs3794271 are detected. Non-limiting examples of such SNPs are found in Table 1.

TABLE 1
Presence of linkage disequilibrium for
the PDE3A-SLCO1C1 locus (rs3794271)

| Polymorphism | Basepair position on | | | |
|---|---|---|---|---|
| ID | chromosome 12 | Allele 1 | Allele 2 | LD |
| rs11045399 | 20,862,939 | G | A | 0.54 |
| rs10770707 | 20,861,451 | A | T | 0.99 |
| rs3794271 | 20,860,093 | G | A | 1.00 |
| rs3838816 | 20,859,984 | AT | A | 0.54 |
| rs1473993 | 20,859,650 | C | T | 0.87 |
| rs10505868 | 20,858,285 | C | T | 0.69 |
| rs78690605 | 20,857,662 | C | CAGAT | 0.69 |
| rs10770706 | 20,857,476 | G | A | 0.69 |
| rs10770705 | 20,857,467 | A | C | 0.53 |
| rs201855778 | 20,857,274 | G | GC | 0.69 |
| rs11392906 | 20,857,273 | T | TC | 0.69 |
| rs10743390 | 20,856,579 | T | C | 0.58 |
| rs71939085 | 20,856,352 | TTGA | T | 0.59 |
| rs10770704 | 20,855,761 | T | C | 0.83 |
| rs10743389 | 20,853,541 | T | C | 0.51 |
| rs10770702 | 20,852,321 | T | G | 0.83 |
| rs959346 | 20,852,086 | G | A | 0.83 |
| rs3751218 | 20,849,067 | G | A | 0.95 |
| rs2417861 | 20,843,580 | C | T | 0.77 |
| rs137927950 | 20,842,088 | T | TTC | 0.66 |
| rs2203493 | 20,841,121 | A | T | 0.75 |
| rs11045392 | 20,840,839 | T | C | 0.84 |
| rs11045390 | 20,840,815 | T | C | 0.85 |
| rs74406912 | 20,840,437 | GT | G | 0.85 |
| rs7305718 | 20,838,164 | C | T | 0.77 |
| rs3809209 | 20,837,585 | C | T | 0.74 |
| rs954866 | 20,834,019 | A | G | 0.85 |
| rs10770701 | 20,832,595 | A | G | 0.81 |
| rs10770699 | 20,832,321 | A | G | 0.51 |
| rs11045385 | 20,831,714 | T | C | 0.62 |
| rs151131008 | 20,830,392 | TTTTTC | T | 0.61 |
| rs10841593 | 20,829,520 | G | A | 0.61 |
| rs12301364 | 20,828,695 | A | G | 0.61 |
| rs10770695 | 20,827,775 | T | C | 0.63 |
| rs9971816 | 20,826,911 | G | A | 0.55 |
| rs11045378 | 20,821,484 | T | A | 0.57 |
| rs11045376 | 20,820,871 | C | T | 0.56 |
| rs10770691 | 20,820,440 | C | G | 0.58 |
| rs6487129 | 20,819,151 | G | A | 0.58 |
| rs10841588 | 20,818,199 | A | G | 0.51 |
| rs10770689 | 20,817,478 | T | C | 0.60 |
| rs10743388 | 20,817,022 | C | G | 0.66 |
| rs4411318 | 20,815,878 | G | T | 0.54 |
| rs7977591 | 20,815,258 | A | G | 0.52 |
| rs4616084 | 20,813,731 | T | C | 0.52 |
| rs11045371 | 20,812,130 | T | C | 0.52 |
| rs4451779 | 20,811,118 | T | G | 0.60 |
| rs10841586 | 20,807,648 | A | G | 0.57 |
| rs10841585 | 20,805,565 | G | A | 0.53 |

In some embodiments, the method provided herein also includes detecting an allele or a complement thereof of one or more polymorphisms (e.g., SNPs), e.g., 1, 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 41, 42, 43, 44, 45, 46, 47, 48, or 49 polymorphisms listed in Table 1 to aid in the selection of an individual with Ps who should receive treatment with an anti-TNFα drug, such as etanercept (ENBREL™), adalimumab (HUMIRA™), infliximab (REMICADE™), golimumab (SIMPONI®), certolizumab pegol (CIMZIA®), ABT-122 (AbbVie, North Chicago, Ill.), pegsunercept, and combinations thereof. In some instances, the method can include detecting an allele or a complement thereof of rs3794271 and an allele or a complement thereof of at least one or more SNPs listed in Table 1, e.g., 1, 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 41, 42, 43, 44, 45, 46, 47, 48, or 49 SNPs. In some cases, the detection of an allele, such as an allele 1 or allele 2 of a specific SNP in Table 1 indicates that an individual is likely to positively respond to an anti-TNFα therapy. Alternatively, the detection of the other allele for that specific SNP indicates that the individual is not likely to positively respond to the anti-TNFα therapy. In some embodiments, the identification or detection of the presence of one or more alleles presented in Table 1 is performed as an alternative to detecting the presence of an allelic variant at rs3794271.

In some embodiments, the presence or absence of a clinical factor, such as, but not limited to, age of the individual, age of psoriasis onset, gender of the individual, family history of psoriasis, disease activity (i.e., disease severity), presence of psoriatic arthritis, nail dystrophy, body mass index (BMI), erythrodermic phenotype (e.g., appearance of inflammatory skin with erythema and scaling), presence of additions skin forms of psoriasis (i.e., guttate, erythrodermic and pustular psoriasis) and combinations thereof is also determined. In some cases, one or more clinical factors, e.g., 1, 2, 3, 4, 5, 6, 7, 8, 9, 10 or more clinical factors are evaluated and used to determine whether the individual is likely to respond to an anti-TNFα therapy.

Disease activity can be measured according to standard scoring indices such as the Psoriasis Area Severity Index (PASI) during the course of treatment, such as, but not limited to, at baseline (at day 0 or treatment or prior to initiating treatment) and at 12-14 weeks during treatment. PASI is a quantitative rating scale for measuring the severity of psoriasis based on the area of skin affected and the appearance of the plaques (e.g., redness, thickness and scaliness of the plaques). The index is calculated as a score that ranges from 0 which corresponds to no disease to 72 which corresponds to maximal disease. Other measurements of disease activity or severity can also be used in this method including the National Psoriasis Foundation (NPF) Psoriasis Score (NPF-PS).

In some instances, Ps patients with at least one G allele at rs3794271 and nail dystrophy have a reduced or lower response to an anti-TNFα therapy compared to those without nail dystrophy. In some cases, Ps patients with at least one G allele at rs3794271 and a high or elevated BMI can have a reduced or lower response to an anti-TNFα therapy compared to those with a lower BMI. In other instances, patients with erythrodermic psoriasis have a lower or decreased response to an anti-TNFα drug compared to those patients without erythrodermic psoriasis.

The method of the present disclosure can also include applying a statistical analysis to the presence of at least one G allele in the PDE3A-SLCO1C1 locus in combination with the presence of one or SNP variants of Table 1, and/or the presence or status of one or more clinical factors of Ps. Detailed descriptions of the statistical analysis are provided below. The statistical analysis can improve the sensitivity, specificity and/or overall accuracy for selecting an individual for treatment with an anti-TNFα drug and/or selecting an anti-TNFα drug for treating psoriasis in an individual in need thereof. The methods disclosed herein have increased sensitivity, specificity, and/or overall accuracy in predicting a positive response to an anti-TNFα drug in a human subject with Ps compared to standard methods in the art such as methods consisting of evaluating or scoring the presence or severity of one or more clinical factors. The methods disclosed herein are advantageous over using disease classification criteria for therapy selection.

B. Therapy Selection for Psoriatic Arthritis

Provided herein is a method for selecting an individual with psoriatic arthritis (PsA) to receive an anti-TNFα therapy. The method includes detecting the presence of an allelic variant (e.g., SNP) in the PDE3A-SLCO1C1 locus in a biological sample obtained from a subject. In some embodiments, if the subject carries two A alleles or complementary alleles thereof (i.e., T alleles) at rs3794271, then the individual should be administered an anti-TNFα therapy for treating PsA. Such an individual is also predicted to have a high likelihood of having a positive response an anti-TNFα therapy.

If it is determined that an individual with PsA carries at least one G allele at rs3794271, then it is predicted that the individual will not have a positive response to an anti-TNF therapy. Rather, such an individual is predicted to have a high likelihood of responding to a therapy that is not an anti-TNFα therapy. As such, provided herein is a method for selecting an individual with PsA to receive a therapy for treating PsA that is not an anti-TNFα therapy. Non-limiting examples of such therapies include ustekinumab (STELARA®), bimekizumab, ixekizumab, sirukumab, sarilumab, secukinumab (COSENTYX®), ocrelizumab, rituximab (Rituxin®), tocilizumab (ACTEMRA®), ofatumumab (ARZERRA®), denosumab (XGEVA®), abatacept (ORENCIA®), masitinib, baricitinib, tofacitinib (XEJLANZ®), anakinra (KINERET®), ABP 501 (Amgen), liraglutide, disease-modifying anti-rheumatic drugs (DMARDs), a nonsteroidal anti-inflammatory drugs (NSAIDs), phosphodiesterase 4 (PDE4) inhibitors, retinoids, glucocorticoids, an immunosuppressive drugs, biosimilars, derivatives thereof, analogs thereof, and any combination thereof.

In other words, an individual with PsA and the genotype of AG or GG at rs3794271 is predicted to have a poor response or no response to an anti-TNFα therapy, such as etanercept (ENBREL™), adalimumab (HUMIRA™), infliximab (REMICADE™), golimumab (SIMPONI®), certolizumab pegol (CIMZIA®), ABT-122 (AbbVie, North Chicago, Ill.), pegsunercept, biosimilars thereof, derivatives thereof, analogs thereof, and any combination thereof. Compared to an individual with an AA genotype at rs3794271, an individual with an AG or GG genotype at the same SNP is expected to have a reduced response to an anti-TNFα drug, yet a positive response to a therapy which can be therapeutically effective for treating PsA but is not an anti-TNFα therapy.

In some embodiments, the method for selecting or recommending treatment for a patient with PsA also includes detecting the presence of one or more SNP alleles set forth in Table 1. In some instances, the method also includes detecting a specific allele (e.g., allele 1 or allele 2) or a complement thereof of one or more polymorphisms (e.g., SNPs), e.g., 1, 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 41, 42, 43, 44, 45, 46, 47, 48, or 49 polymorphisms listed in Table 1. The presence of the specific allele (e.g., allele 1 or allele 2) can indicate that the individual who also has PsA should be administered a therapy that is not an anti-TNFα therapy, such as biological inhibitors of either IL6, IL6 receptor, IL12 (e.g., IL12 or p40 subunit of IL12), IL23 (e.g., p19 subunit of IL23 or p40 subunit of IL23), IL17A, IL-17F, IL17 receptor A, CD20, or a combination thereof, small molecule inhibitors that do no inhibit, block or target TNFα, DMARDs that are not anti-TNFα drugs, nonsteroidal anti-inflammatory drugs, PDE4 inhibitors, retinoids, glucocorticoids, immunosuppressive drugs, biosimilars thereof, derivatives thereof, and analogs thereof.

In some instances, the method includes detecting at least one allele or a complement thereof at rs3794271 in addition to a specific allele (e.g., allele 1 or allele 2) or a complement thereof of one or more SNPs, e.g., 1, 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 41, 42, 43, 44, 45, 46, 47, 48, or 49 SNPs listed in Table 1. In some embodiments, the method includes detecting the presence of, for example, allele 1 or a complement thereof of at least one SNP of Table 1, e.g., 1, 2, 3, 4, 5, 6, 7, 8, 9, 10, 11, 12, 13, 14, 15, 16, 17, 18, 19, 20, 25, 30, 35, 40, 45 or 49 SNPs of Table 1 instead of detecting the presence of an allele or a complement thereof at rs3794271. Alternatively, the method includes detecting the presence of, for example, allele 2 or a complement thereof of at least one SNP of Table 1, e.g., 1, 2, 3, 4, 5, 6, 7, 8, 9, 10, 11, 12, 13, 14, 15, 16, 17, 18, 19, 20, 25, 30, 35, 40, 45 or 49 SNPs of Table 1 instead of detecting the presence of an allele or a complement thereof at rs3794271.

In some instances, the method additionally includes detecting the presence, status or level of one or more clinical factors, such as, but not limited to, the age of the individual, gender of the individual, disease duration, HLA-B27 genotype, presence/severity of dactylitis (e.g., inflammation or swelling of an entire digit), presence/severity of enthesitis (e.g., inflammation or swelling at sites where tendons, ligaments, joint capsules or fascia attach to the bone), presence of axial involvement, presence/severity of nail dystrophy, severity of the skin disease and the severity of psoriatic arthritis. In preferred embodiments, the clinical factor is gender, e.g., male or female. In some cases, the presence, status or level of 1, 2, 3, 4, 5, 6, 7, 8, 9, 10 or more clinical factors are determined and factored together using an algorithm or statistical analysis.

The severity of the skin disease can be assessed as mild or moderate-to-severe according to the level of the body surface area (BSA) affection. For instance, a patient with mild skin disease may have <10% BSA at the time of sample collection or during the course of the disease. A patient with moderate-to-severe skin disease may have a higher level of BSA (e.g., >10% BSA).

The severity of PsA can be evaluated by a clinician by using the CASPAR classification criteria for PsA (Taylor et al., Arth Rheum, 2006, 54:2665-2673). To satisfy the criteria for having PsA, a patient has inflammatory articular disease of joint, spine or entheseal with at least 3 points from the following features: current psoriasis (scores 2 points), a personal or family history of psoriasis if no current psoriasis, nail dystrophy, negative RF, dactylitis and juxta-articular new bone formation.

The method of the present disclosure can include applying a statistical analysis to the presence of a G allele (i.e., one G allele or two G alleles) or the complementary allele thereof or two AA alleles at rs3794271 in the PDE3A-SLCO1C1 locus in combination with the presence of one or SNP variants of Table 1, and/or the presence of one or more clinical factors.

C. Methods of Genotyping

A variety of means can be used to genotype an individual at a polymorphic site in a gene or any other genetic marker described herein to determine whether a sample (e.g., a nucleic acid sample) contains a specific variant allele or haplotype. For example, enzymatic amplification of nucleic acid from an individual can be conveniently used to obtain nucleic acid for subsequent analysis. The presence of a specific variant allele or haplotype in one or more genetic markers of interest can also be determined directly from the individual's nucleic acid without enzymatic amplification. In preferred embodiments, an individual is genotyped at the PDE3A-SLCO1C1 locus.

Genotyping of nucleic acid from an individual, whether amplified or not, can be performed using any of various techniques. Useful techniques include, without limitation, polymerase chain reaction (PCR) based analysis, sequence analysis, and electrophoretic analysis, which can be used alone or in combination. As used herein, the term “nucleic acid” means a polynucleotide such as a single- or double-stranded DNA or RNA molecule including, for example, genomic DNA, cDNA and mRNA. This term encompasses nucleic acid molecules of both natural and synthetic origin as well as molecules of linear, circular, or branched configuration representing either the sense or antisense strand, or both, of a native nucleic acid molecule. It is understood that such nucleic acids can be unpurified, purified, or attached, for example, to a synthetic material such as a bead or column matrix.

Material containing nucleic acid is routinely obtained from individuals. Such material is any biological matter from which nucleic acid can be prepared. As non-limiting examples, material can be whole blood, serum, plasma, synovial fluid, saliva, cheek swab, sputum, or other bodily fluid or tissue that contains nucleic acid. In one embodiment, a method of the present invention is practiced with whole blood, which can be obtained readily by non-invasive means and used to prepare genomic DNA. In another embodiment, genotyping involves amplification of an individual's nucleic acid using the polymerase chain reaction (PCR). Use of PCR for the amplification of nucleic acids is well known in the art (see, e.g., Mullis et al. (Eds.), The Polymerase Chain Reaction, Birkhäuser, Boston, (1994)). In yet another embodiment, PCR amplification is performed using one or more fluorescently labeled primers. In a further embodiment, PCR amplification is performed using one or more labeled or unlabeled primers that contain a DNA minor groove binder.

Any of a variety of different primers can be used to amplify an individual's nucleic acid by PCR in order to determine the presence of a variant allele in one or more genes or loci or other genetic marker in a method of the invention. Non-limiting examples of loci include the PDE3A-SLCO1C1 locus on chromosome 12p12.2. For example, the PCR primers can be used to amplify specific regions of the PDE3A-SLCO1C1 locus, such as the polymorphism at rs3794271. As understood by one skilled in the art, additional primers for PCR analysis can be designed based on the sequence flanking the polymorphic site(s) of interest in the PDE3A-SLCO1C1 locus or other genetic marker (e.g., SNP variants of Table 1). As a non-limiting example, a sequence primer can contain from about 15 to about 30 nucleotides of a sequence upstream or downstream of the polymorphic site of interest in the PDE3A-SLCO1C1 locus or other genetic marker. Such primers generally are designed to have sufficient guanine and cytosine content to attain a high melting temperature which allows for a stable annealing step in the amplification reaction. Several computer programs, such as Primer Select, are available to aid in the design of PCR primers.

A Taqman® allelic discrimination assay available from Applied Biosystems can be useful for genotyping an individual at a polymorphic site and thereby determining the presence of a particular variant allele or haplotype in the PDE3A-SLCO1C1 locus, or other genetic marker (e.g., SNP variants of Table 1). In a Taqman® allelic discrimination assay, a specific fluorescent dye-labeled probe for each allele is constructed. The probes contain different fluorescent reporter dyes such as FAM and VIC to differentiate amplification of each allele. In addition, each probe has a quencher dye at one end which quenches fluorescence by fluorescence resonance energy transfer. During PCR, each probe anneals specifically to complementary sequences in the nucleic acid from the individual. The 5′ nuclease activity of Taq polymerase is used to cleave only probe that hybridizes to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Those skilled in the art understand that improved specificity in allelic discrimination assays can be achieved by conjugating a DNA minor groove binder (MGB) group to a DNA probe as described, e.g., in Kutyavin et al., Nuc. Acids Research 28:655-661 (2000). Minor groove binders include, but are not limited to, compounds such as dihydrocyclopyrroloindole tripeptide (DPI3).

Sequence analysis can also be useful for genotyping an individual according to the methods described herein to determine the presence of a particular variant allele or haplotype in the PDE3A-SLCO1C1 locus (e.g., r53794271) or other genetic marker (e.g., SNP variants of Table 1). As is known by those skilled in the art, a variant allele of interest can be detected by sequence analysis using the appropriate primers, which are designed based on the sequence flanking the polymorphic site of interest in the PDE3A-SLCO1C1 locus, or another genetic marker. Additional or alternative sequence primers can contain from about 15 to about 30 nucleotides of a sequence that corresponds to a sequence about 40 to about 400 base pairs upstream or downstream of the polymorphic site of interest in one or more of the PDE3A-SLCO1C1 locus, or another genetic marker. Such primers are generally designed to have sufficient guanine and cytosine content to attain a high melting temperature which allows for a stable annealing step in the sequencing reaction.

The term “sequence analysis” includes any manual or automated process by which the order of nucleotides in a nucleic acid is determined. As an example, sequence analysis can be used to determine the nucleotide sequence of a sample of DNA. The term sequence analysis encompasses, without limitation, chemical and enzymatic methods such as dideoxy enzymatic methods including, for example, Maxam-Gilbert and Sanger sequencing as well as variations thereof. The term sequence analysis further encompasses, but is not limited to, sequencing by synthesis (Loman et al., Nature Biotech, 2012, 30:434-439), other next-generation sequencing platforms (see, e.g., Mardis, Ann Rev Anal Chem, 2013, 6:287-303), capillary array DNA sequencing, which relies on capillary electrophoresis and laser-induced fluorescence detection and can be performed using instruments such as the MegaBACE 1000 or ABI 3700. As additional non-limiting examples, the term sequence analysis encompasses thermal cycle sequencing (see, Sears et al., Biotechniques 13:626-633 (1992)); solid-phase sequencing (see, Zimmerman et al., Methods Mol. Cell Biol. 3:39-42 (1992); and sequencing with mass spectrometry, such as matrix-assisted laser desorption/ionization time-of-flight mass spectrometry (see, MALDI-TOF MS; Fu et al., Nature Biotech. 16:381-384 (1998)). The term sequence analysis further includes, but is not limited to, sequencing by hybridization (SBH), which relies on an array of all possible short oligonucleotides to identify a segment of sequence (see, Chee et al., Science 274:610-614 (1996); Drmanac et al., Science 260:1649-1652 (1993); and Drmanac et al., Nature Biotech. 16:54-58 (1998)). One skilled in the art understands that these and additional variations are encompassed by the term sequence analysis as defined herein.

Electrophoretic analysis also can be useful in genotyping an individual according to the methods of the present invention to determine the presence of a particular variant allele or haplotype in the PDE3A-SLCO1C1 locus, or another genetic marker. “Electrophoretic analysis” as used herein in reference to one or more nucleic acids such as amplified fragments includes a process whereby charged molecules are moved through a stationary medium under the influence of an electric field. Electrophoretic migration separates nucleic acids primarily on the basis of their charge, which is in proportion to their size, with smaller molecules migrating more quickly. The term electrophoretic analysis includes, without limitation, analysis using slab gel electrophoresis, such as agarose or polyacrylamide gel electrophoresis, or capillary electrophoresis. Capillary electrophoretic analysis generally occurs inside a small-diameter (50-100 m) quartz capillary in the presence of high (kilovolt-level) separating voltages with separation times of a few minutes. Using capillary electrophoretic analysis, nucleic acids are conveniently detected by UV absorption or fluorescent labeling, and single-base resolution can be obtained on fragments up to several hundred base pairs. Such methods of electrophoretic analysis, and variations thereof, are well known in the art, as described, for example, in Ausubel et al., Current Protocols in Molecular Biology Chapter 2 (Supplement 45) John Wiley & Sons, Inc. New York (1999).

Restriction fragment length polymorphism (RFLP) analysis can also be useful for genotyping an individual according to the methods of the present invention to determine the presence of a particular variant allele or haplotype in the PDE3A-SLCO1C1 locus or other genetic marker (see, Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, “restriction fragment length polymorphism analysis” includes any method for distinguishing polymorphic alleles using a restriction enzyme, which is an endonuclease that catalyzes degradation of nucleic acid following recognition of a specific base sequence, generally a palindrome or inverted repeat. One skilled in the art understands that the use of RFLP analysis depends upon an enzyme that can differentiate a variant allele from a wild-type or other allele at a polymorphic site.

In addition, allele-specific oligonucleotide hybridization can be useful for genotyping an individual in the methods described herein to determine the presence of a particular variant allele or haplotype in the PDE3A-SLCO1C1 locus, or another genetic marker (e.g., SNP variant in Table 1). Allele-specific oligonucleotide hybridization is based on the use of a labeled oligonucleotide probe having a sequence perfectly complementary, for example, to the sequence encompassing the variant allele. Under appropriate conditions, the variant allele-specific probe hybridizes to a nucleic acid containing the variant allele but does not hybridize to the one or more other alleles, which have one or more nucleotide mismatches as compared to the probe. If desired, a second allele-specific oligonucleotide probe that matches an alternate (e.g., wild-type) allele can also be used. Similarly, the technique of allele-specific oligonucleotide amplification can be used to selectively amplify, for example, a variant allele by using an allele-specific oligonucleotide primer that is perfectly complementary to the nucleotide sequence of the variant allele but which has one or more mismatches as compared to other alleles (Mullis et al., supra). One skilled in the art understands that the one or more nucleotide mismatches that distinguish between the variant allele and other alleles are often located in the center of an allele-specific oligonucleotide primer to be used in the allele-specific oligonucleotide hybridization. In contrast, an allele-specific oligonucleotide primer to be used in PCR amplification generally contains the one or more nucleotide mismatches that distinguish between the variant and other alleles at the 3′ end of the primer.

A heteroduplex mobility assay (HMA) is another well-known assay that can be used for genotyping in the methods of the present invention to determine the presence of a particular variant allele or haplotype in the PDE3A-SLCO1C1 locus, or another genetic marker. HMA is useful for detecting the presence of a variant allele since a DNA duplex carrying a mismatch has reduced mobility in a polyacrylamide gel compared to the mobility of a perfectly base-paired duplex (see, Delwart et al., Science, 262:1257-1261 (1993); White et al., Genomics, 12:301-306 (1992)).

The technique of single strand conformational polymorphism (SSCP) can also be useful for genotyping in the methods described herein to determine the presence of a particular variant allele or haplotype in the PDE3A-SLCO1C1 locus, or another genetic marker (see, Hayashi, Methods Applic., 1:34-38 (1991)). This technique is used to detect variant alleles based on differences in the secondary structure of single-stranded DNA that produce an altered electrophoretic mobility upon non-denaturing gel electrophoresis. Variant alleles are detected by comparison of the electrophoretic pattern of the test fragment to corresponding standard fragments containing known alleles.

Denaturing gradient gel electrophoresis (DGGE) can also be useful in the methods of the invention to determine the presence of a particular variant allele or haplotype in the PDE3A-SLCO1C1 locus, or another genetic marker. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (see, Sheffield et al., “Identifying DNA Polymorphisms by Denaturing Gradient Gel Electrophoresis” in Innis et al., supra, 1990).

Other molecular methods useful for genotyping an individual are known in the art and useful in the methods of the present invention. Such well-known genotyping approaches include, without limitation, automated sequencing and RNase mismatch techniques (see, Winter et al., Proc. Natl. Acad. Sci., 82:7575-7579 (1985)). Furthermore, one skilled in the art understands that, where the presence of multiple variant alleles is to be determined, individual variant alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple variant alleles can be detected in individual reactions or in a single reaction (a “multiplex” assay).

In view of the above, one skilled in the art realizes that the methods of the present invention for providing information regarding the likelihood of response to treatment with an anti-TNFα therapy in patients with Ps or PsA, and for providing information regarding the selection of a suitable therapeutic regimen for the treatment of Ps or PsA (e.g., by determining the presence of one or more variant alleles of genes such as, but not limited to, the PDE3A-SLCO1C1 locus) can be practiced using one or any combination of the well-known genotyping assays described above or other assays known in the art.

D. Statistical Analysis

In some aspects, the present invention provides methods for determining the likelihood of response to treatment with anti-TNFα therapy by detecting the presence of one or more variant alleles (e.g., SNPs) at the PDE3A-SLCO1C1 locus, alone or in combination with detecting the presence of one or more SNPs in linkage disequilibrium with the SNP of the PDE3A-SLCO1C1 locus, and/or the presence of one or more clinical factors (e.g., patient age, sex, etc.), and applying a statistical analysis such as quantile analysis or a learning statistical classifier system to the genotype(s) detected at the PDE3A-SLCO1C1 locus and optionally to the presence of one or more SNP variants, and/or to the presence of one or more clinical factors. In certain embodiments, the use of statistical analyses in the methods of the present invention advantageously provide improved sensitivity, specificity, negative predictive value, positive predictive value, and/or overall accuracy for predicting or identifying the probability that a patient with Ps or PsA will respond to treatment with one or more anti-TNFα therapies, compared to other methods used for selecting a treatment regimen such as a clinical criteria.

The term “statistical analysis” or “statistical algorithm” or “statistical process” includes any of a variety of statistical methods and models used to determine relationships between variables. In the present invention, the variables are the presence, level, or genotype of at least one marker of interest. Any number of markers can be analyzed using a statistical analysis described herein. For example, the presence or level of 1, 2, 3, 4, 5, 6, 7, 8, 9, 10, 11, 12, 13, 14, 15, 16, 17, 18, 19, 20, 25, 30, 35, 40, 45, 50, 55, 60, or more markers can be included in a statistical analysis. In one embodiment, logistic regression is used. In another embodiment, linear regression is used. In certain preferred embodiments, the statistical analyses comprise a quantile measurement of one or more markers, e.g., within a given population, as a variable. Quantiles are a set of “cut points” that divide a sample of data into groups containing (as far as possible) equal numbers of observations. For example, quartiles are values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The present invention can also include the use of percentile ranges of marker levels (e.g., tertiles, quartile, quintiles, etc.), or their cumulative indices (e.g., quartile sums of marker levels to obtain quartile sum scores (QSS), etc.) as variables in the statistical analyses (just as with continuous variables).

In some embodiments, the statistical analyses comprise one or more learning statistical classifier systems. As used herein, the term “learning statistical classifier system” includes a machine learning algorithmic technique capable of adapting to complex data sets (e.g., panel of markers of interest) and making decisions based upon such data sets. In some embodiments, a single learning statistical classifier system such as a decision/classification tree (e.g., random forest (RF) or classification and regression tree (CART)) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (CART), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as nave learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ).

Random forests are learning statistical classifier systems that are constructed using an algorithm developed by Leo Breiman and Adele Cutler. Random forests use a large number of individual decision trees and decide the class by choosing the mode (i.e., most frequently occurring) of the classes as determined by the individual trees. Random forest analysis can be performed, e.g., using the RandomForests software available from Salford Systems (San Diego, Calif.). See, e.g., Breiman, Machine Learning, 45:5-32 (2001); and at the website stat-www.berkeley.edu/users/breiman/RandomForests/cc_home.htm, for a description of random forests.

Classification and regression trees represent a computer intensive alternative to fitting classical regression models and are typically used to determine the best possible model for a categorical or continuous response of interest based upon one or more predictors. Classification and regression tree analysis can be performed, e.g., using the CART software available from Salford Systems or the Statistica data analysis software available from StatSoft, Inc. (Tulsa, Okla.). A description of classification and regression trees is found, e.g., in Breiman et al. “Classification and Regression Trees,” Chapman and Hall, New York (1984); and Steinberg et al., “CART: Tree-Structured Non-Parametric Data Analysis,” Salford Systems, San Diego, (1995).

Neural networks are interconnected groups of artificial neurons that use a mathematical or computational model for information processing based on a connectionist approach to computation. Typically, neural networks are adaptive systems that change their structure based on external or internal information that flows through the network. Specific examples of neural networks include feed-forward neural networks such as perceptrons, single-layer perceptrons, multi-layer perceptrons, backpropagation networks, ADALINE networks, MADALINE networks, Learnmatrix networks, radial basis function (RBF) networks, and self-organizing maps or Kohonen self-organizing networks; recurrent neural networks such as simple recurrent networks and Hopfield networks; stochastic neural networks such as Boltzmann machines; modular neural networks such as committee of machines and associative neural networks; and other types of networks such as instantaneously trained neural networks, spiking neural networks, dynamic neural networks, and cascading neural networks. Neural network analysis can be performed, e.g., using the Statistica data analysis software available from StatSoft, Inc. See, e.g., Freeman et al., In “Neural Networks: Algorithms, Applications and Programming Techniques,” Addison-Wesley Publishing Company (1991); Zadeh, Information and Control, 8:338-353 (1965); Zadeh, “IEEE Trans. on Systems, Man and Cybernetics,” 3:28-44 (1973); Gersho et al., In “Vector Quantization and Signal Compression,” Kluywer Academic Publishers, Boston, Dordrecht, London (1992); and Hassoun, “Fundamentals of Artificial Neural Networks,” MIT Press, Cambridge, Mass., London (1995), for a description of neural networks.

Support vector machines are a set of related supervised learning techniques used for classification and regression and are described, e.g., in Cristianini et al., “An Introduction to Support Vector Machines and Other Kernel-Based Learning Methods,” Cambridge University Press (2000). Support vector machine analysis can be performed, e.g., using the SVM^{light} software developed by Thorsten Joachims (Cornell University) or using the LIBSVM software developed by Chih-Chung Chang and Chih-Jen Lin (National Taiwan University).

As used herein, the term “sensitivity” refers to the probability that a predictive method of the present invention gives a positive result when the sample is positive, e.g., having the predicted therapeutic response to an anti-TNFα therapy in an individual with Ps or PsA. Sensitivity is calculated as the number of true positive results divided by the sum of the true positives and false negatives. Sensitivity essentially is a measure of how well the present invention correctly identifies those with Ps or PsA who have the predicted therapeutic response to an anti-TNFα therapy. The statistical methods and models can be selected such that the sensitivity is at least about 60%, and can be, e.g., at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “specificity” refers to the probability that a predictive method of the present invention gives a negative result when the sample is not positive, e.g., not having the predicted therapeutic response to an anti-TNF therapy in an individual with Ps or PsA. Specificity is calculated as the number of true negative results divided by the sum of the true negatives and false positives. Specificity essentially is a measure of how well the present invention excludes those with Ps or PsA who do not have the predicted therapeutic response to an anti-TNF therapy. The statistical methods and models can be selected such that the specificity is at least about 60%, and can be, e.g., at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

As used herein, the term “negative predictive value” or “NPV” refers to the probability that an individual identified as not having the predicted therapeutic response to an anti-TNF therapy. Negative predictive value can be calculated as the number of true negatives divided by the sum of the true negatives and false negatives. Negative predictive value is determined by the characteristics of the diagnostic or prognostic method as well as the prevalence of the disease in the population analyzed. The statistical methods and models can be selected such that the negative predictive value in a population having a disease prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “positive predictive value” or “PPV” refers to the probability that an individual identified as having the predicted therapeutic response to an anti-TNF therapy. Positive predictive value can be calculated as the number of true positives divided by the sum of the true positives and false positives. Positive predictive value is determined by the characteristics of the predictive method as well as the prevalence of the disease in the population analyzed. The statistical methods and models can be selected such that the positive predictive value in a population having a disease prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Predictive values, including negative and positive predictive values, are influenced by the prevalence of the disease in the population analyzed. In the present invention, the statistical methods and models can be selected to produce a desired clinical parameter for a clinical population with a particular Ps or PsA prevalence. For example, statistical methods and models can be selected for a Ps or PsA prevalence of up to about 1%, 2%, 3%, 4%, 5%, 6%, 7%, 8%, 9%, 10%, 15%, 20%, 25%, 30%, 35%, 40%, 45%, 50%, 55%, 60%, 65%, or 70%, which can be seen, e.g., in a clinician's office such as a rheumatologist's office or a general practitioner's office.

As used herein, the term “overall agreement” or “overall accuracy” refers to the accuracy with which a method of the present invention predicts response to an anti-TNFα therapy. Overall accuracy is calculated as the sum of the true positives and true negatives divided by the total number of sample results and is affected by the prevalence of the disease in the population analyzed. For example, the statistical methods and models can be selected such that the overall accuracy in a patient population having a disease prevalence is at least about 40%, and can be, e.g., at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

E. Anti-TNFα Therapies

Anti-TNFα therapies such as the anti-TNFα drugs disclosed herein can be administered to a patient with Ps or PsA in a therapeutically effective amount for treating one or more symptoms associated with Ps or PsA, respectively. The present invention advantageously enables a clinician to practice “precision medicine” by guiding treatment decisions and informing therapy selection for Ps or PsA such that a specific therapeutically effective drug treatment can be administered to a subject in need thereof. Provided herein is a method for treating Ps or PsA that includes administering an anti-TNFα therapy such as, but not limited to, etanercept (ENBREL™), adalimumab (HUMIRA™), infliximab (REMICADE™), golimumab (SIMPONI®), certolizumab pegol (CIMZIA®), ABT-122 (AbbVie, North Chicago, Ill.), pegsunercept (Amgen, Thousand Oaks, Calif.), biosimilars thereof, derivatives thereof, analogs thereof, and any combination thereof.

Any of the anti-TNFα therapies described herein can be administered alone to a Ps patient or in combination with another type of therapy for treating Ps. Similarly, the anti-TNFα therapies described herein can be administered alone to a PsA patient or in combination with another type of therapy for treating PsA.

F. Other Therapies for Treating Ps or PsA that are not Anti-TNFα Therapies

Provided herein is a method for treating a subject with Ps or PsA that includes administering to the subject a therapeutically effective amount of a therapy for treating Ps or PsA, respectfully, that is not an anti-TNFα therapy (e.g., a non-TNFα therapy).

Examples of such therapies for treating Ps include, but are not limited to, corticosteroids, vitamin D analogues, retinoids, anthralin, calcineurin inhibitors, salicylic acid, coal tar, phototherapy, e.g., ultraviolet light therapy, UVB phototherapy, narrow band UVB therapy, Goeckerman therapy, photochemotherapy, and excimer laser therapy, methotrexate, cyclosporine, thioguanine, hydrourea, ustekinumab (STELARA®), A3 adenosine receptor agonists, anti-IL-17 agents, anti-IL-12/23 agents, anti-IL-17 receptor agents, Janus kinase (JAK) inhibitors, phosphodiesterase 4 (PDE4) inhibitors, biosimilars thereof, analogs thereof, derivative thereof, and combinations thereof. Additional examples of a therapy for treating Ps that is not an anti-TNFα therapy include acitretin, alefacept (AMEVIVE®), photocil, tofacitinib, memotasone, brodalumab, efalizumab, guselkumab, secukinumab, siplixumab, ustekinumab, toreforant, psoralait, tinefcon, apremilast, cilomilast, diazepam, ibudilast, luteolin, mesembrenone, piclamilast, roflumilast and rolipram, and combinations thereof.

Examples of therapies for treating PsA that are not an anti-TNFα therapy include, but are not limited to, ustekinumab (STELARA®), bimekizumab, ixekizumab, sirukumab, sarilumab, secukinumab (COSENTYX®), ocrelizumab, rituximab (Rituxin®), tocilizumab (ACTEMIRA®), ofatumumab (ARZERRA®), denosumab (XGEVA®), abatacept (ORENCIA®), masitinib, baricitinib, tofacitinib (XEJLANZ®), anakinra (KINERET®), ABP 501 (Amgen), liraglutide, disease-modifying anti-rheumatic drugs (DMARDs), nonsteroidal anti-inflammatory drugs (NSAIDs), PDE4 inhibitors, retinoids, glucocorticoids, immunosuppressive drugs, biosimilars thereof, derivatives thereof, analogs thereof, and any combination thereof. A therapy for Ps or PsA that is not an anti-TNFα therapy can be a DMARD, NSAID, PDE4 inhibitor, biological inhibitor that does not inhibit or antagonize TNFα, biosimilars thereof, analogs thereof, derivatives thereof, or any combination thereof.

Non-limiting examples of DMARDs include methotrexate, leflunomide, D-penicillamine, azathioprine, gold salts (e.g., sodium aurothiomalate, auranofin, etc.), cyclosporine, minocycline, anti-malarial medications (e.g., chloroquine, hydroxychloroquine, sulfasalazine, etc.), free bases thereof, pharmaceutically acceptable salts thereof, derivatives thereof, analogs thereof, and combinations thereof.

Examples of NSAIDs include, but are not limited to, ibuprofen, indomethacin, COX-2 inhibitors (e.g., celecoxib), free bases thereof, pharmaceutically acceptable salts thereof, derivatives thereof, analogs thereof, and combinations thereof.

Non-limiting examples of PDE4 inhibitors include apremilast, cilomilast, diazepam, ibudilast, luteolin, mesembrenone, piclamilast, roflumilast, rolipram, and combinations thereof.

Any one of the therapies described herein that is useful for treating Ps can be administered to a Ps patient alone or in combination with one or more other therapies for treating Ps. Similarly, any of the therapies that is useful for treating PsA can be administered to a PsA patient alone or in combination with one or more other therapies for treating PsA.

G. Dosages and Methods of Administration

Anti-TNFα therapies or non-TNFα therapies disclosed herein can be administered with a suitable pharmaceutical excipient as necessary and can be carried out via any of the accepted modes of administration. Thus, administration can be, for example, intravenous, topical, subcutaneous, transcutaneous, transdermal, intramuscular, oral, buccal, sublingual, gingival, palatal, intra joint, parenteral, intra-arteriole, intradermal, intraventricular, intracranial, intraperitoneal, intralesional, intranasal, rectal, vaginal, or by inhalation. By “co-administer” it is meant that an anti-TNFα biological inhibitor is administered at the same time, just prior to, or just after the administration of a second drug (e.g., another anti-TNFα biological inhibitor drug, a drug useful for reducing the side-effects of the first drug, etc.).

A therapeutically effective amount of an anti-TNFα drug or a non-anti-TNFα drug provided herein may be administered repeatedly, e.g., at least 2, 3, 4, 5, 6, 7, 8, or more times, or the dose may be administered by continuous infusion. The dose may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for human subjects and other mammals, each unit containing a predetermined quantity of, e.g., an anti-TNFα drug calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of, e.g., the anti-TNFα drug.

Methods for preparing such dosage forms are known to those skilled in the art (see, e.g., Remington's Pharmaceutical Sciences, 18^{th} ed., Mack Publishing Co., Easton, Pa. (1990)). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., Remington's Pharmaceutical Sciences, supra). Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the therapeutically effective dose takes the form of a pill, tablet, or capsule, and thus, the dosage form can contain, along with, e.g., an anti-TNFα drug, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. An anti-TNFα therapy can also be formulated into a suppository disposed, for example, in a polyethylene glycol (PEG) carrier.

Liquid dosage forms can be prepared by dissolving or dispersing, e.g., an anti-TNFα drug and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. For topical administration, the therapeutically effective dose can be in the form of emulsions, lotions, gels, foams, creams, jellies, solutions, suspensions, ointments, and transdermal patches. For administration by inhalation, for example, an anti-TNFα drug can be delivered as a dry powder or in liquid form via a nebulizer. For parenteral administration, the therapeutically effective dose can be in the form of sterile injectable solutions and sterile packaged powders. Preferably, injectable solutions are formulated at a pH of from about 4.5 to about 7.5.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

An anti-TNFα drug or a non-TNFα drug, such as a PDE4 inhibitor, can be administered at the initial dosage of from about 0.001 mg/kg to about 1000 mg/kg daily. A daily dose range of from about 0.01 mg/kg to about 500 mg/kg, from about 0.1 mg/kg to about 200 mg/kg, from about 1 mg/kg to about 100 mg/kg, or from about 10 mg/kg to about 50 mg/kg, can be used. The dosages, however, may be varied depending upon the requirements of the individual, the severity of Ps symptoms, and the anti-TNFα biological inhibitor drug being employed. For example, dosages can be empirically determined considering the severity of psoriasis symptoms, the stage of Ps, and/or the prognosis of Ps in an individual. The dose administered to an individual, in the context of the present invention, should be sufficient to affect a beneficial therapeutic response in the individual over time. The size of the dose can also be determined by the existence, nature, and extent of any adverse side-effects that accompany the administration of a particular anti-TNFα biological inhibitor drug in an individual. Determination of the proper dosage for a particular situation is within the skill of the practitioner. Generally, treatment is initiated with smaller dosages which are less than the optimum dose of the anti-TNFα drug or the non-anti-TNFα drug. Thereafter, the dosage is increased by small increments until the optimum effect under circumstances is reached. For convenience, the total daily dosage may be divided and administered in portions during the day, if desired.

### IV. Examples

The present invention will be described in greater detail by way of a specific example. The following example is offered for illustrative purposes, and is not intended to limit the invention in any manner. Those of skill in the art will readily recognize a variety of noncritical parameters which can be changed or modified to yield essentially the same results.

### Example 1. Association of the PDE3A-SLCO1C1 Locus with the Response to Anti-TNFα Agents in Psoriasis

Psoriasis is a prevalent autoimmune disease of the skin that causes significant psychological and physical disability. Tumor Necrosis Factor α (TNF α) blocking agents have proven to be highly efficacious in the management of moderate to severe psoriasis. However, a significant percentage of patients do not respond to this treatment. Recently, variation at PDE3A-SLCO1C1 locus has been robustly associated to anti-TNFα response in rheumatoid arthritis. Using a cohort of 130 psoriasis patients treated with anti-TNFα therapy, we sought to analyze the association of this locus with treatment response in psoriasis. We found a highly significant association between PDE3A-SLCO1C1 and the clinical response to TNFα blockers (P=0.0031). Importantly, the allele that was previously associated with the lack of response to rheumatoid arthritis (G allele at r53794271) was associated with a higher anti-TNFα efficacy in psoriasis. The results of this study are an important step in the characterization of the pharmacogenetic profile associated with anti-TNFα response in psoriasis.

Genome-wide association studies (GWAS) provide a powerful approach for the analysis of the genetic risk associated to multiple traits. GWAS can detect new candidate genes associated with treatment response without the need for prior biological knowledge. Recently, a GWAS of anti-TNFα response in rheumatoid arthritis in the Danish population identified a large group of candidate regions (Krintel et al., Pharmacogenet Genomics 2012; 22(8): 577-589). Using an independent cohort of rheumatoid arthritis patients from the Spanish population we were able to replicate the association between PDE3A-SLCO1C1 locus on chromosome 12p12.2 and anti-TNFα response (Acosta-Colman et al. Pharmacogenomics 2013; 14(7): 727-734). Combining the evidence between the two patient cohorts, PDE3A-SLCO1C1 association was the first pharmacogenetic locus to reach the genome-wide level of significance in rheumatoid arthritis. Although the genetic basis of rheumatoid arthritis and psoriasis susceptibility is markedly different, we sought to test if the variation at this genomic region is also associated with the response to anti-TNFα therapy in psoriasis.

Using a cohort of 130 patients, we have analyzed the association between the PDE3A-SLCO1C1 locus chromosome 12p12.2 and the clinical response to anti-TNFα therapy in psoriasis. Additionally, we have analyzed the influence of different clinical factors on the genetic association to treatment response. The work disclosed was published as Julia et al., The Pharmacogenomics Journal, 2014, 15:322-325.

### Patients and Methods

### Patients

A total of 130 patients with psoriasis who underwent treatment with an anti-TNFα agent (etanercept, adalimumab or infliximab) were included in this study. The patients were recruited from the outpatients clinics of the dermatology departments from 11 university hospitals from Spain participating in the IMID Consortium (Julia et al., Hum Mol Genet 2012; 21(20): 4549-4557). Psoriasis patients with plaque psoriasis affecting torso and/or extremities and with at least one year of duration were included. Patients with a single clinical localization of plaque psoriasis (i.e., scalp, face, palmoplantar), with exclusively inverse plaque psoriasis or with an inflammatory bowel disease were excluded from the study.

All patients received anti-TNFα treatment as their first biologic therapy after having a refractory psoriasis to ≧1 conventional systemic therapy, including methotrexate, cyclosporine, acitretin, or phototherapy. Disease activity was measured at baseline and at 12-14 weeks of treatment using the Psoriasis Area and Severity Index (PASI) (Fredriksson T, Pettersson U., Dermatologica 1978; 157(4): 238-244).

### Genotyping

Whole blood samples were obtained from all patients and genomic DNA was extracted using the Chemagic Magnetic Separation Module (Perkin Elmer, USA). All samples were processed and stored at the IMID-Biobank (Vall Hebron Research Institute, Spain) until analysis. Genotyping of the PDE3A-SLCO1C1 locus single nucleotide polymorphism (SNP) rs3794271 was performed using the Real Time TaqMan® PCR genotyping system (Life Technologies, Carlsbad, Calif.). The C_27502188_10 Taqman® SNP genotyping assay was used. RT-PCR thermal conditions were as follows: 50° C. for 2 min and 95° C. for 10 min, followed by 40 cycles of 92° C. for 15 s and 60° C. for 1 min. The PCR assay and point fluorescent readings were performed using an ABI PRISM′ 79001-IT sequence detection system (Life Technologies). 20% (n=26) of the individuals were genotyped in duplicate to estimate genotyping error. The regenotyping of these samples was performed using the same technological platform and the same DNA aliquot as the initial genotyping. All genotypes were replicated and thus we estimated a <1% genotyping; error in the study.

### Statistical Analysis

Response to therapy was measured using the relative change in the PASI between baseline and at 12-14 weeks of anti-TNFα therapy: PASI response=(PASI_{baseline}−PASI_{endpoint})/PASI_{baseline}×100). The PASI response measures the percentage of improvement in disease activity relative to a specified baseline (Julia et al., JAMA Dermatol 2013; 149(9): 1033-1039). The use of a relative score has the added advantage that it avoids any potential bias associated with different levels of disease activity at baseline. The association of the rs3794271 SNP genetic variation with the PASI response was performed using linear regression.

Clinical variables including age, age at psoriasis onset, duration of psoriasis, gender, presence of psoriatic arthritis, body-mass index (BMI), nail involvement, family history of psoriasis, severity of disease, presence of additional skin forms of psoriasis (i.e., guttate, erythrodermic and pustular psoriasis) and type of anti-TNF agent were also tested for association with the response to anti-TNF treatment. Severity of disease was determined as described previously (Julia et al., Hum Mol Genet 2012; 21(20): 4549-4557). Briefly, psoriasis patients are categorized into mild or moderate-severe disease according to the maximum level of Body Surface Area (BSA) affection achieved from disease diagnosis to the time of recruitment in the study. Patients never reaching a BSA of 10% were categorized into having a mild disease and patients having had ≧10% BSA were included in the moderate-severe group. The association between each variable and the PASI response was tested using linear regression. All statistical analyses were performed using the R statistical software version 3.0.1 (Team RC (2013). R: A Language and Environment for Statistical Computing. (R Foundation for Statistical Computing, Vienna, 2009).

The present study was performed following the Declaration of Helsinki protocols, and all patients gave their written informed consent to participate in this study. The study was approved by the local Institutional Review Boards of each of the participating centers.

### Results

A total of 130 psoriasis patients having received an anti-TNF therapy were analyzed in the present study. Table 2 summarizes the main clinical features of the patient cohort.

TABLE 2
Clinical characteristics of the psoriasis patient cohort

| Clinical Variable | Summary statistic | |
|---|---|---|
| Age at study entry (mean years, SD) | 44.9 | (13.9) |
| Age at psoriasis onset (mean years, SD) | 23.6 | (12.5) |
| Duration of psoriasis (mean years, SD) | 21.4 | (13.1) |
| Gender (n male, %) | 92 | (70.7%) |
| Psoriatic arthritis (n, %) | 19 | (14.6%) |
| BMI (mean kg/m², SD) | 47.1 | (8.3) |
| Nail involvement (n, %) | 71 | (54.6%) |
| Family history of psoriasis (n, %) | 66 | (50.7%) |
| Severity of disease (n moderate-severe, %) | 120 | (92.3%) |
| Additional psoriasis forms: | | |
| Guttate Psoriasis (n, %) | 17 | (13.1%) |
| Erythrodermic Psoriasis (n, %) | 9 | (6.9%) |
| Pustular Psoriasis (n, %) | 2 | (1.5%) |
| Anti-TNF agent | | |
| Etanercept (n, %) | 78 | (60%) |
| Adalimumab (n, %) | 36 | (27.7%) |
| Infliximab (n, %) | 16 | (12.3%) |
| Baseline PASI (mean, SD) | 20.2 | (10.1) |
| Endpoint PASI (mean, SD) | 3.8 | (5.0) |
| PASI response (mean, SD) | 80.1% | (22.4) |

We found a highly significant association between the genetic variation at PDE3A-SLCO1C1 SNP rs3794271 and the response to anti-TNFα treatment in patients with psoriasis (P=0.0031). Compared with the genetic association in rheumatoid arthritis, the minor allele of rs3794271 SNP (G allele) was associated with an improvement of the clinical response to anti-TNFα therapy. Patients carrying 1 or 2 copies of the minor allele G had, on average, >10% improvement in the PASI response compared to patients homozygous for the major allele A (t-test for difference in means between the two groups P=0.00017). Table 3 shows the average PASI response according to each rs3794271 SNP genotype.

TABLE 3
Genotype frequencies and associated PASI response
for PDE3A-SLCO1C1 locus at rs3794271

| | | Mean (SD) PASI | | | | |
|---|---|---|---|---|---|---|
| Genotype | N | response | Freq | MAF | Beta | P-value |
| AA | 59 | 73.1% (26.3) | 0.45 | 0.33 | 8.5 | 0.0031 |
| AG | 56 | 85.8% (17.9) | 0.43 | | | |
| GG | 15 | 86.3% (10.7) | 0.12 | | | |

SD: standard deviation; Freq: genotype frequency; Beta: regression coefficient of PDE3A-SLCO1C1 SNP; P-value: significance of association of rs3794271 SNP with anti-TNF response in psoriasis.

Analyzing the association of the clinical variables as potential predictors of anti-TNFα therapy, we found a significant association between nail involvement, body-mass-index (BMI) and erythrodermic psoriasis with the PASI response (P=0.021, P=0.05 and P=0.018, respectively). Patients with nail disease were found to have a lower response to anti-TNFα therapy (mean±SD PASI response with nail involvement=76.1%±25.1, without nail involvement=85.3%±17.7). We also found that BMI was negatively correlated with the PASI response (r²=−0.17, P_{correlation}=0.05). The presence of erythrodermic psoriasis also was associated with a lower response to anti-TNFα therapy (mean±SD PASI response with erythrodermic psoriasis=63.1%±34.3, without erythrodermic psoriasis=81.4%±20.9). No other clinical variables, including anti-TNFα treatment type, showed a significant association with the clinical response. In order to control for the potential effect of the covariates on the observed PDE3A-SLCO1C1 association with anti-TNF response, we used a multivariate linear regression analysis. Including all significant clinical variables in the multivariate model, the association between rs3794271 SNP and the PASI response remained highly significant (P=0.00057).

Finally, to evaluate the trend of the association of rs3794271 genotype and positive clinical response according to anti-TNFα treatment type, we performed a stratified analysis. We found a significant association of the SNP with PASI response with the group of etanercept-treated patients (P=0.012) and a trend in infliximab-treated patients (P=0.14).

### Discussion

Psoriasis is a highly heterogeneous disease characterized by a wide variety of phenotypic manifestations and degrees of severity. In those cases with highest severity and that are refractory to other systemic therapies, anti-TNFα agents are a powerful and highly effective therapeutic option. However, in 20-40% of psoriasis patients, TNFα blockade is not sufficient to control the disease activity in the skin. Consequently, there is an unmet need to identify markers that can help predict the type of response to anti-TNFα agents in psoriasis. In the present study we have found a significant association between the PDE3A-SLCO1C1 locus with the clinical response to anti-TNFα agents in psoriasis. The results of this study are an important step in the personalization of anti-TNFα therapy in psoriasis and provide new important clues on the clinical and molecular features that differentiate anti-TNFα responsive patients from non-responders.

PDE3A-SLCO1C1 locus SNP rs3794271 lies in the fourth intron of the SLCO1C1 gene. Extensive genotype data of this locus on chromosome 12p12.2 indicates that this SNP lies in a linkage disequilibrium region that includes both the 3′ terminal region of PDE3A gene and the promoter and 5′ region of SLCO1C1 gene (Acosta-Colman et al., Pharmacogenomics 2013; 14(7): 727-734). Therefore, the genetic variant causing the association could be related to any of the two genes. Since none of the coding variants at the PDE3A or SLCO1C1 genes is in strong linkage disequilibrium with rs3794271 SNP, it is likely that the genetic variation associated with anti-TNF response in psoriasis participates in a genetic regulatory process. Using recent expression quantitative trait loci data generated by the sequencing of the transcriptome of lymphoblastoid cell lines (Lappalainen et al., Nature 2013; 501(7468): 506-511), we did not find any significant evidence of cis or trans gene expression regulation for this SNP. It is likely, however, that the regulatory functionality of the PDE3A-SLCO1C1 locus associated with the response to anti-TNFα therapy in psoriasis is cell-type specific and will only be identified in the cells relevant to the disease. Consequently, future functional experiments on specific skin and immune cell types should clarify the specific role of this genetic region in the response to anti-TNFα therapy.

There is biological evidence supporting the role of both PDE3A and SLCO1C1 genes in drug response. Phosphodiesterase 3A (PDE3A) belongs to a large family of cyclic nucleotide phosphodiesterases (PDEs) that catalyze the hydrolysis of cAMP and cGMP into the inactive forms 5′-AMP and 5′-GMP, respectively. PDEs participate in multiple intercellular signaling processes that affect multiple cell subtypes and biological functions. Phosphodiesterase 4D (PDE4D) is strongly associated to the immune and inflammatory activity and, importantly, therapies targeting this protein have shown to be efficacious in the treatment of psoriasis (Schafer P., Biochem Pharmacol 2012; 83(12): 1583-1590). SoLute Carrier Organic anion transporter family member 1C1 gene (SLCO1C1) belongs to the organic-anion transporting polypeptide (OATP) family of cell membrane transporters. This group of proteins has been associated to the active transport of different organic molecules, toxins and drugs. Members of this family have been shown to be constitutively expressed in human keratinocytes (Schiffer et al., J Invest Dermatol 2003; 120(2): 285-291). Importantly, there is sound evidence demonstrating that polymorphisms in SLCO1B1, a member of the OATP family, have strong pharmacogenetic effects on different types of drugs including methotrexate, a treatment for psoriasis and a drug that is generally coadministered with anti-TNF agents to reduce immunogenicity (Ramsey et al., Blood 2013; 121(6): 898-904). Deep sequencing of the PDE3A-SLCO1C1 genomic region in anti-TNF responder and non-responder psoriasis patients will help to determine the precise DNA variation associated with treatment response and, subsequently, perform functional studies to determine the biological activity associated with this genomic region.

In the present study we have also found that the presence of nail involvement, increased BMI and the erythrodermic phenotype are bad prognosis factors for the response to anti-TNFα agents in psoriasis. There is increasing evidence that BMI has a strong influence in different aspects of psoriasis epidemiology (Carrascosa et al., Actas Dermosifiliogr 2014; 105(1): 31-44; Puig L., J Eur Acad Dermatol Venereol 2011; 25(9): 1007-1011). Importantly, there is evidence that overweight is associated with a loss of efficacy of anti-TNFα agents (Carrascosa et al., J Eur Acad Dermatol Venereol 2014; 28(7):907-14). This study confirms the finding and supports the importance of an adequate management of obesity in the treatment of psoriasis. To our knowledge, this study provides the first evidence that nail involvement and the erythrodermic phenotype are bad prognosis factors for anti-TNFα treatment. Finally, including the associated clinical variables as covariates, we found that PDE3A-SLCO1C1 association with the PASI response is still strong. This result demonstrates that the newly identified genetic association between PDE3A-SLCO1C1 and anti-TNF response in psoriasis is little influenced by clinical variability.

The minor allele of the PDE3A-SLCO1C1 locus SNP rs3794271 (G) was associated with a significant reduction of the disease activity in the psoriasis skin, while it was associated with a worse response to anti-TNFα agents in rheumatoid arthritis. In rheumatoid arthritis, the most common disease activity score, the DAS28 (Fransen and van Riel, Rheum Dis Clin North Am 2009; 35(4): 745-757, vii-viii), is a composite score measures the degree of inflammation in the synovial joints and at the systemic level. Therefore, the results suggest that while blocking TNFα is an effective means of controlling the inflammatory activity in psoriasis and rheumatoid arthritis, the biological mechanisms that underlie both types of treatment response are markedly different. There is substantial evidence that the cell-type features that characterize the psoriasis lesion with the rheumatoid arthritis synovial inflammation are clearly different (Bowcock A M, Krueger J G., Nat Rev Immunol 2005; 5(9): 699-711). Furthermore, in some rheumatoid arthritis patients, TNFα blocking has shown to induce psoriasis (Sfikakis et al., Arthritis Rheum 2005; 52(8): 2513-2518; Harrison et al., Ann Rheum Dis 2009; 68(2): 209-215.), clearly supporting the existence of very different pathogenic mechanisms. Recently, pharmacogenetic studies of FCGR2A and FCGR3A gene variants and anti-TNFα therapy response have provided suggestive evidence for opposite genetic effects between rheumatoid arthritis and Crohn's Disease (Moroi et al., Immunogenetics 2013; 65(4): 265-271), rheumatoid arthritis and psoriatic arthritis (Ramirez et al., J Rheumatol 2012; 39(5): 1035-1041) and, even between rheumatoid arthritis and psoriasis (Julia et al., JAMA Dermatol 2013; 149(9): 1033-1039). Together, these results support the existence of different TNFα-related pathways and, consequently, different biologic mechanisms by which TNFα blocking is an effective treatment.

Stratifying by the type of anti-TNFα agents, we found that the genetic association was statistically significant in the group of psoriasis patients treated with etanercept. Also, when all three anti-TNFα agents (etanercept, adalimumab and infliximab) were analyzed together, a significant association between the PDE3A-SLCO1C1 locus SNP rs3794271 and positive clinical response. The result suggest that the association with psoriasis is not specific to a single agent.

In this study we have found an association between the PDE3A-SLCO1C1 locus and the response to anti-TNFα therapy in psoriasis. The allele associated with a positive response in psoriasis is the same allele associated with a lack of clinical response in rheumatoid arthritis, suggesting the existence of large biological differences at the molecular level, despite the similar efficacy of TNF blockers. The results of this study are an important step in the translational research in psoriasis and will clearly lead to new research lines that will improve the prognosis of psoriasis patients with more severe disease.

### Example 2. Variation at PDE3A-SLCO1C1 Locus is Associated with Poor Response to Anti-TNFα Therapy in Psoriatic Arthritis

Variation at PDE3A-SLCO1C1 locus has been recently associated with the response to anti-TNFα therapy in rheumatoid arthritis. We undertook the present study to determine whether PDE3A-SLCO1C1 is also associated with the response to anti-TNFα therapy in psoriatic arthritis. Genomic DNA was obtained from eighty-one psoriatic arthritis patients that had been treated with anti-TNFα therapy. PDE3A-SLCO1C1 SNP rs3794271 was genotyped using TaqMan® real time-PCR. The clinical response to anti-TNFα therapy was measured as the change from baseline in the level of disease activity according to the DAS28 score. A significant association between the rs3794271 SNP and reduced or poor response to anti-TNFα therapy in psoriatic arthritis was found (beta=−0.71; p=0.0036). In addition, a significant interaction between PDE3A-SLCO1C1 locus and gender was identified (p=0.033). The results show that the G allele at a SNP in the PDE3A-SLCO1C1 locus is associated with a poor response to anti-TNFα drug therapy in subjects with psoriatic arthritis compared to PsA subject with an AA genotype at rs3794271. The observed pharmacogenetic association in PsA shows a strong gender-specific effect. This work has been published in Julia et al., Pharmacogenomics, 2014, 15(14):1763-1769.

### Methods

### Patient Population

A total of eighty-one patients were recruited for the present study. All patients were collected in the framework of the immune-Mediated Inflammatory Disease Consortium (Acosta-Colman et al., Pharmacogenomics 14(7), 727-734 (2013)). PsA patients were recruited through the outpatients' clinics of the rheumatology departments from 26 university hospitals from Spain.

The inclusion criteria were as follows: fulfillment of the PsA CASPAR classification criteria; >18 years old; Caucasian and born in Spain, all four grandparents born in Spain; ≧1 year of disease evolution; having received an anti-TNFα therapy (e.g., adalimumab, etanercept or infliximab) as the first biological treatment; and baseline Disease Activity Score for 28 joints (DAS28 score) ≧3.2. The exclusion criteria were as follows: rheumatoid factor positivity; presence of any other inflammatory joint disease; and presence of inflammatory bowel disease. The presence of axial disease was determined using the modified New York criteria (Van Der Linden et al., Arthritis Rheum, 1984, 27(4):361-368). Informed consent was obtained from all participants and protocols were reviewed and approved by the local institutional review boards. All procedures were performed in accordance with the Declaration of Helsinki.

### Treatment Response Definition and Statistical Power Estimation

The clinical response was measured as the absolute change in Disease Activity Score for 28 joints (DAS28) between weeks 0 and 12 of anti-TNFα treatment (i.e. ΔDAS28). The DAS28 score is used extensively to evaluate disease activity in PsA patients (Wagner et al., Ann Rheum Dis 72(1), 83-88 (2013)). It combines a 28 swollen joint count, a 28 tender joint count, erythrocyte sedimentation rate, and a patient global assessment into a continuous index. The ΔDAS28 score has an approximate normal distribution and is commonly used in pharmacogenetic studies (Cui et al., Arthritis Rheum 62(7), 1849-1861 (2010); Murdaca et al., J Invest Dermatol, (2014)). In order to estimate the statistical power of our sample size, we used Quanto software (v 1.2.4) (Gauderman W J, Morrison J M: QUANTO 1.1: A computer program for power and sample size calculations for genetic-epidemiology studies. University of Southern California, Department of Biostatistics, (2006)). An additive inheritance model was assumed and rs3794271 SNP minor allele frequency was obtained from the study sample.

### Genotyping

Whole blood samples were obtained from all 81 PsA patients and genomic DNA was extracted using the Chemagic Magnetic Separation Module I (Perkin Elmer, USA). The PDE3A-SLCO1C1 locus single nucleotide polymorphism (SNP) rs3794271 was genotyped in the cohort of PsA patients. Genotyping was performed using the TaqMan® real-time PCR platform (Life Technologies, US) using the C_27512188_10 assay (Acosta-Colman et al., Pharmacogenomics 14(7), 727-734 (2013)). Thermal conditions were as follows: 50° C. for 2 min and 95° C. for 10 min, followed by 40 cycles of 92″C for 15 s and 60° C. for 1 min.. The PCR assay and point fluorescent readings were performed using an ABI PRISPe 7900HT sequence detection system (Life Technologies). The genotyping error was determined by re-genotyping 20% of the patient cohort.

### Genetic and Clinical Variables Association Analysis

The association of the PDE3A-SLCO1C1 SNP rs3794271 with a response to anti-TNFα therapy was performed using linear regression. Briefly, the rs3794271 genotype in each individual was codified as 0, 1 or 2 according to the number of minor (G) alleles. The regression coefficient was then estimated using the least squares filling method. The response to therapy was measured using the absolute change in the DAS28 between baseline and week 12 of anti-TNFα therapy (ΔDAS28) (Fransen J, Van Riel P L, Rheum Dis Clin North Am 35(4), 745-757, vii-viii (2009)). Similar to previous studies, the baseline DAS28 was included as a covariate in the linear regression model (Acosta-Colman et al., Pharmacogenomics 14(7), 727-734 (2013); Krintel et al., Pharmacogenet Genomics 22(8), 577-589 (2012)). Additionally, multiple linear regression was also used to evaluate the different clinical variables as predictors of the response to anti-TNF therapy either alone or in interaction with rs3794271 genotype. Tested variables included gender, age, disease duration, HLA-B27 genotype, dactylitis, enthesitis, axial involvement, nail dystrophy and severity of skin disease. The latter was defined as described previously (Julia et al., Hum Mol Genet 21(20), 4549-4557 (2012)). Briefly, severity of skin disease is defined as either mild or moderate-to-severe according to the level of Body Surface Area (BSA) affection. Mild skin affection is defined as those patients that have never reached >10% of BSA at the time of sample collection nor in their clinical record. PsA patients showing higher levels of BSA were classified as moderate-to-severe psoriasis.

The interaction of each clinical variable with the PDE3A-SLCO1C1 locus SNP was analyzed by including an interaction term to the linear regression model as previously described (Julia et al., Hum Mol Genet 21(20), 4549-4557 (2012)). The presence of a drug type-specific effect (i.e., specific interaction with adalimumab, etanercept or infliximab) was evaluated as described previously (Plant et al., Arthritis Rheum 63(3), 645-653 (2011)). All association analyses were performed using the R statistical software version 3.0.1.

### Results

Using a cohort of 81 PsA patients we tested the association of PDE3A-SLCO1C1 SNP rs3794271 with the response to anti-TNFα therapy. Table 4 shows a detailed summary of the clinical and epidemiological characteristics of the patient cohort used in this study. The call rate for rs3794271 was >99% and the genotyping error was <1%. The study sample size had a power of >80% to detect a difference in DAS28 as low as 0.57 units for the rs3794271 minor allele frequency (MAF)=0.27.

TABLE 4
Characteristics of the Psoriatic Arthritis
(PsA) patients analyzed in this study

| Variable | Summary | |
|---|---|---|
| Female, n (%) | 38 | (47%) |
| Mean (SD) age, years | 48.9 | (12.7) |
| Mean (SD) PsA duration, years | 9.1 | (8.2) |
| HLA-B27 positive, n (%) | 11 | (13.6%) |
| Dactylitis, n (%) | 18 | (22.2%) |
| Enthesitis, n (%) | 2 | (2.4%) |
| Axial involvement, n (%) | 18 | (22.2%) |
| Nail dystrophy, n (%) | 43 | (53.1%) |
| Moderate-Severe skin disease, n/%) | 26 | (32.1%) |
| Mean (SD) baseline DAS28 | 5.27 | (0.97) |
| Mean (SD) DAS28 at wk 14 | 3.19 | (1.27) |
| Anti-TNFα therapy | | |
| Etanercept, n (%) | 34 | (42%) |
| Infliximab, n (%) | 21 | (26%) |
| Adalimumab, n (%) | 26 | (32%) |

DAS28, 28-joint Disease Activity Score; HLA: Human leukocyte antigen.

We found a highly significant association between the variation at the PDE3A-SLCO1C1 locus and the absolute change in DAS28 score (Beta=−0.71, p=0.0036). Like for RA, the presence of the rs3794271 minor allele C was associated with a reduced response to anti-TNFα treatment compared to an AA genotype at rs3794271. Table 5 shows the mean DAS28 score change for each genotype of the PDE3A-SLCO1C1 locus SNP.

TABLE 5
Genotype frequencies and associated 28-joint count disease
activity score changes for PDE3A-SLCO1C1 rs3794271 SNP

| | | ΔDAS28 Mean | | | | |
|---|---|---|---|---|---|---|
| Genotype | N | (SD) | Freq | MAF | Beta | P-value |
| AA | 39 | 2.42 (1.4) | 0.48 | 0.27 | −0.71 | 0.0036 |
| AG | 40 | 1.82 (1.2) | 0.49 | | | |
| GG | 2 | 0.81 (1.7) | 0.02 | | | |

ΔDAS28: Change in Disease Activity Score in 28 joints; SD: standard deviation; Freq: genotype frequency; Beta: regression coefficient for genotype adjusting for basal DAS28 value; P-value; significance of association of rs3794271 SNP with the response in PsA adjusting for the baseline DAS28 score.

None of the clinical and epidemiological variables analyzed showed a significant association such as a p-value <0.05 with ΔDAS2S between baseline and week 12 of anti-TNFα treatment. Only gender showed a trend towards association with treatment response in the PsA cohort (p=0.072). Gender was also the only clinical variable showing a significant interaction with PDE3A-SLCO1C1 locus SNP genotype (p=0.034) in the association with reduced treatment response.

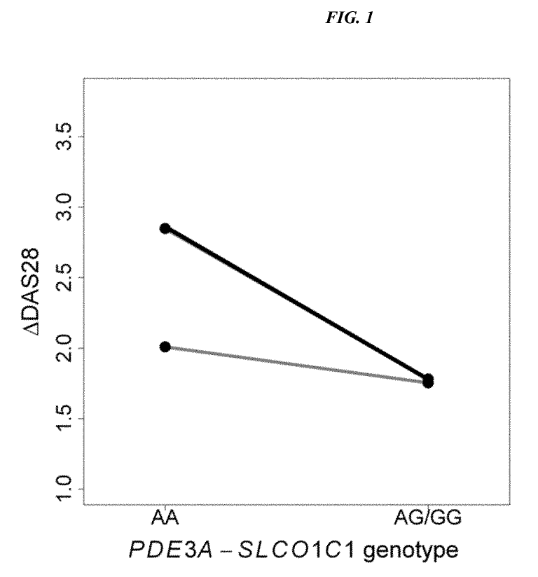

Given the significant interaction between gender and rs3794271 genotype, we investigated the association of the SNP with treatment response in each gender separately. We found a highly significant association between PDE3A-SLCO1C1 locus in male patients (Beta=−1.2, p=0.00034) and treatment response. The analysis in the female patient subgroup, however, did not show a significant association between the SNP and treatment response (Beta=−0.19, p=0.59). Directly comparing the effect sizes of the association in each gender subgroup (Beta in the linear model), we found a statistically significant difference (p=0.034, FIG. 1).

In order to rule out the effect of confounders in the observed differential genetic association, we also compared the distribution of all clinical and epidemiological variables between both genders. In this comparison, only disease duration showed a statistically significant difference between the male and female patient subgroups (mean (SD) years males=8.81 (5.83), mean (SD) years females 14.22 (9.53), t-test for difference in means p=0.0044). Consequently, we re-evaluated the association of PDE3A-SLCO1C1 locus with anti-TNFα response within each gender including the disease duration as covariate to control for its potential effect. Adjusting for this variable, however, the association between rs3794271 and anti-TNFα response in each gender subgroup remained unaltered (p=0.00075 and p=0.26 for male and female subgroups, respectively), thus discarding any potential confounding effect of disease duration in the observed genetic association.

### Discussion

The response to anti-TNFα therapy in PsA patients is variable. For instance some patients show significant positive clinical response while other patients show little to no positive clinical response due to inefficient TNF α blockage to control the chronic inflammatory process. There is a need for the identification of treatment response markers that can be used to predict or forecast treatment response and thus, assist in the clinical management of PsA.

We recently have identified a genetic variation in the PDE3A-SLCO1C1 locus at chromosome 12p12.2 associated with reduced response to anti-TNFα response in RA (Acosta-Colman et al., Pharmeogenomics, 14(7):727-734 (2013)). Given the similar pathology between the two diseases, we hypothesized that the PDE3A-SLCO1C1 locus could also be associated with a poor response to an anti-TNFα therapy in patients with PsA. Using a cohort of 81 patients treated with anti-TNFα therapy, we have found compelling evidence that a genetic variation at this locus (e.g., a G allele at r53794271) is associated with poor treatment response in PsA patients.

The PDE3A-SLCO1C1 SNP rs3794271 lies in a linkage disequilibrium region that encompasses the 3′ end of phosphodiesterase 3A gene (PDE3A) as well as the promoter region and 5′ region of the SoLute Carrier Organic anion transporter family member 1C1 gene (SLCO1C1) (Fredriksson T, Pettersson U., Dermatologica 1978; 157(4): 238-244). PDE3A belongs to the family of phosphodiesterases (PDEs) that catalyze the hydrolysis of cyclic AMP and cyclic GMP, thereby regulating their intracellular concentrations and their signaling pathways (Puig L., J Eur Acad Dermatol Venereal 2011; 25(9): 1007-1011). Several inflammatory pathways, including the TNF alpha cytokine pathway, have been shown to be regulated by cAMP and cGMP signaling systems. Consequently, PDEs have been studied as targets for treating diseases with aberrant immune responses like PsA. For example, apremilast, a second generation phosphodiesterase 4D (PDE4D inhibitor, has shown good results in Phase II and Phase III trials in psoriasis and PsA (Carrascosa et al., J Eur Acad Dermatol Venereal 2014), and it has very recently been approved by the U.S. Food and Drug Administration for the treatment of PsA. There is also evidence that inhibiting PDE3A could be a useful anti-inflammatory therapy. The PDE3A inhibitor Cilostazol® (Pletal; Otsuka Pharmaceutical) has shown to have anti-inflammatory effects in different cells and tissues (Bowcock A M, Krueger J G., Nat Rev Immunol 2005; 5(9): 699-711) and it has even shown to suppress synovial inflammation and joint destruction in RA mouse models (Sfikakis et al., Arthritis Rheum 2005; 52(8): 2513-2518). Therefore, this study suggests that inhibiting PDE3A could also be a useful therapeutic strategy in PsA.

SLCO1C1 belongs to the organic anion transporting polypeptides (OATP) family. This group of proteins is associated with the transport of drugs into cells and, consequently, they are also likely biological candidates for influencing treatment response in PsA (Harrison et al., Ann Rheum Dis 2009; 68(2): 209-215). The human Organic Anion-Transporting Polypeptide 1B1 (SLCO1B1), for example, has been shown to influence the disposition of methotrexate (MTX) which is the treatment of choice for PsA when non-steroidal anti-inflammatory drugs or local glucocorticoid injections fail to control the disease activity (Sun et al., Nat Genet 2010; 42(11): 1005-1009). Furthermore, genetic variation at SLCO1B1 gene has been shown to influence MTX clearance and treatment response (Moroi et al., Immunogenetics 2013; 65(4): 265-271). Given that anti-TNF therapies in PsA are commonly used in combination with MTX, the variation at SLCO1C1 could also influence the disposition of this drug and, consequently, the overall efficacy of the therapy. Future studies analyzing the association between the genetic variation at PDE3A-SLCO1C1 locus and the variation of the disposition of both drugs in PsA are warranted.

Like in RA, the minor allele G of rs3794271 SNP is associated with a worse or reduced response to anti-TNFα therapy in subjects with PsA compared to PsA subjects with two A alleles at rs3794271. In both diseases the same treatment response definition was used which measures the disease activity in joints. In PsA, however, there is also a skin component and anti-TNFα agents have shown also to be highly beneficial in the management of psoriasis (Sun et al., Nat Genet 2010; 42(11): 1005-1009).

We did not find drug-type specific effects in PDE3A-SLCO1C1 association with anti-TNFα response in PsA patients. Instead, we found a significant interaction between gender and the observed genetic association. In the previous study, gender did not show a significant interaction with rs3794271 SNP in RA patients.

However, based on the results of this study, we further analyzed the association between PDE3A-SLCO1C1 and treatment response in female RA patients and male RA patients separately. While the genetic association with anti-TNFα response was significant in both genders, the effect size was stronger in the male subgroup compared to the female subgroup. Combined, this evidence suggests that the biological pathway in which the PDE3A-SLCO1C1 locus participates has distinctive regulatory features according to gender.

### CONCLUSIONS

This example describes an association of the PDE3A-SLCO1C1 locus with the response to anti-TNFα therapy such as etanercept, adalimumab, or infliximab in PsA patients. The results of this study show that a reduced or poor response to anti-TNFα therapy in RA and PsA patients is influenced by a common genetic variation, i.e., an AG or GG genotype at rs3794271. The data also show that patients with PsA and an AA genotype at rs3794271 have a high likelihood of positively responding to an anti-TNFα therapy. Such patients have an increased likelihood of clinically responding to an anti-TNFα compared to patients with an AG or GG genotype at the same SNP. As such, patients suffering from PsA and having an AA genotype at rs37944271 should be selected/recommended for, or administered etanercept, adalimumab, infliximab, or another anti-TNFα drug for the treatment of PsA.

We have also identified a strong interaction between the variation at PDE3A-SLCO1C1 and gender in treatment response, which highlights the importance of accounting for clinical and epidemiological variation in pharmacogenetic studies. We identified a significant difference between the strength of association of the PDE3A-SLCO1C1 polymorphism in male PsA patients compared to female patients. The present study represents the identification of a new genetic biomarker of therapeutic response to anti-TNFα therapy in PsA. Consequently, this is an important step in the personalization of treatment decisions for this common disease.

In summary, anti-TNFα treatment has been shown to be a highly successful approach for the management of moderate-to-severe psoriatic arthritis. Despite its efficacy, a substantial percentage of psoriatic arthritis patients (about >40%) will not respond to systemic TNFα inhibition or blockade. Clinical variables or factors have been ineffective at predicting, distinguishing or discriminating between responders and non-responders to anti-TNFα therapy. The present study demonstrates that a genetic marker in the PDE3A-SLCO1C locus of chromosome 12p12 can be used to identify non-responders to anti-TNFα therapy prior to drug administration. Using a cohort of 81 PsA patients, we found a statistically significant association between the rs3794271 SNP in the PDE3A-SLCO1C locus and response to anti-TNFα therapy. PsA patients having two A alleles at rs3794271 are predicted to positively respond to an anti-TNFα drug, such as etanercept, adalimumab, infliximab, and other anti-TNFα biological inhibitors. Similar to the findings in RA patients, the presence of a G allele (e.g., one G allele or two G alleles) at rs3794271 in a PsA patient is associated with a lower, reduced or decreased level of response to an anti-TNFα drug compared to a PsA patient having a homozygous A genotype (e.g., AA genotype) at rs3794271. Additionally, a significant difference between the strength of association of the PDE3A-SLCO1C SNP in male PsA patients compared to female patients.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for selecting an individual with psoriasis (Ps) to receive an anti-tumor necrosis factor α (anti-TNFα) therapy, the method comprising:
(a) detecting the presence of a single nucleotide polymorphism (SNP) in a PDE3A-SLCO1C1 locus in a sample obtained from the individual, wherein the SNP in the PDE3A-SLCO1C1 locus comprises rs3794271; and
(b) selecting the individual to receive the anti-TNFα therapy based on the presence of a G allele or a complementary allele thereof at rs3794271.

2. The method of claim 1, wherein selecting the individual to receive the anti-TNFα therapy is based on the presence of two G alleles or the complementary alleles thereof at rs3794271.

3. The method of claim 1, wherein the anti-TNFα therapy is selected from the group consisting of infliximab (REMICADE™), etanercept (ENBREL™), adalimumab (HUMIRA™), certolizumab pegol (CIMZIA®), golimumab (SIMPONI®), ABT-122, pegsunercept, biosimilars thereof, and combinations thereof.

4. The method of claim 1, further comprising detecting the presence of an allele of one or more SNPs in Table 1.

5. The method of claim 1, further comprising detecting the presence of a clinical factor for the individual.

6-11. (canceled)

12. A method for predicting a likelihood of response to an anti-TNFα therapy in an individual with psoriasis (Ps), the method comprising:
(a) detecting the presence of a single nucleotide polymorphism (SNP) in a PDE3A-SLCO1C1 locus in a sample obtained from the individual, wherein the SNP in the PDE3A-SLCO1C1 locus comprises rs3794271; and
(b) determining that the individual has a high likelihood of response to the anti-TNFα therapy based on the presence of a G allele or a complementary allele thereof at rs3794271.

13. The method of claim 12, wherein determining that the individual has the high likelihood of response to the anti-TNFα therapy is based on the presence of two G alleles or the complementary alleles thereof at rs3794271.

14. The method of claim 12, wherein the anti-TNFα therapy is selected from the group consisting of infliximab (REMICADE™), etanercept (ENBREL™), adalimumab (HUMIRA™), certolizumab pegol (CIMZIA®), golimumab (SIMPONI®), ABT-122, pegsunercept, biosimilars thereof, and combinations thereof.

15. The method of claim 12, further comprising detecting the presence of an allele of one or more SNPs in Table 1.

16. The method of claim 12, further comprising detecting the presence of a clinical factor for the individual.

17-22. (canceled)

23. A method for selecting an individual with psoriatic arthritis (PsA) to receive an anti-TNFα therapy, the method comprising:
(a) detecting the presence of a single nucleotide polymorphism (SNP) in a PDE3A-SLCO1C1 locus in a sample obtained from the individual, wherein the SNP in the PDE3A-SLCO1C1 locus comprises rs3794271; and
(b) selecting the individual to receive the anti-TNFα therapy based on the presence of two A alleles or the complementary alleles thereof at rs3794271.

24. The method of claim 23, wherein the anti-TNFα therapy is selected from the group consisting of infliximab (REMICADE™), etanercept (ENBREL™), adalimumab (HUMIRA™), certolizumab pegol (CIMZIA®), golimumab (SIMPONI®), ABT-122, pegsunercept, biosimilars thereof, and combinations thereof.

25. The method of claim 23, further comprising detecting the presence of an allele of one or more SNPs in Table 1.

26. The method of claim 23, further comprising detecting the presence of a clinical factor for the individual.

27-32. (canceled)

33. A method for predicting a likelihood of response to an anti-TNFα therapy in an individual with psoriatic arthritis (PsA), the method comprising:
(a) detecting the presence of a single nucleotide polymorphism (SNP) in a PDE3A-SLCO1C1 locus in a sample obtained from the individual, wherein the SNP in the PDE3A-SLCO1C1 locus comprises rs3794271; and
(b) determining that the individual has a high likelihood of response to the anti-TNFα therapy based on the presence of two A alleles or the complementary alleles thereof at rs3794271.

34. The method of claim 33, wherein the anti-TNFα therapy is selected from the group consisting of infliximab (REMICADE™), etanercept (ENBREL™), adalimumab (HUMIRA™), certolizumab pegol (CIMZIA®), golimumab (SIMPONI®), ABT-122, pegsunercept, biosimilars thereof, and combinations thereof.

35. The method of claim 33, further comprising detecting the presence of an allele of one or more SNPs in Table 1.

36. The method of claim 33, further comprising detecting the presence of a clinical factor for the individual.

37-52. (canceled)
